(19) United States
(12) Patent Application Publication
(10) Pub. No.: US 2022/0193220 A1
(43) Pub. Date: Jun. 23, 2022

(54) IMMUNOGENIC COMPOSITIONS FOR NOVEL REASSORTANT MAMMALIAN ORTHEOVIRUS FROM PIGS

(71) Applicants: Kansas State University Research Foundation, Manhattan, KS (US); Centers for Disease Control and Prevention, Washington, DC (US)

(72) Inventors: Wenjun Ma, Manhattan, KS (US); Liping Wang, Manhattan, KS (US); Suxiang Tong, Atlanta, GA (US)

(21) Appl. No.: 17/594,921

(22) PCT Filed: May 4, 2020
(86) PCT No.: PCT/US20/31376

§ 371 Date: Nov. 3, 2021

Related U.S. Application Data
(60) Provisional application No. 62/843,150, filed on May 3, 2019.

(51) Int. Cl. A61K 39/12; C12N 7/00; A61P 31/14

(52) U.S. Cl. CPC A61K 39/12; A61K 2039/53; A61P 31/14; C12N 7/00

(57) ABSTRACT

An immunogenic composition for reducing the incidence or severity of subclinical and clinical signs of orthoreovirus infection is provided. The composition(s) includes at least one segment or portion thereof of orthoreovirus that is derived from a different serotype, host, or strain than at least one other segment or portion thereof. The present disclosure also provides methods for treating, preventing, and reducing the subclinical and clinical signs of orthoreovirus infection in a subject or group of subjects.

### CROSS REFERENCE TO RELATED APPLICATIONS

The present application claims the priority benefit of Provisional Application Ser. No. 62/843,150, filed on May 3, 2019, the teachings and contents of which are hereby incorporated by reference in their entirety.

### BACKGROUND OF THE DISCLOSURE

The field of the disclosure relates generally to Mammalian orthoreovirus (MRV). MRV is a double-stranded RNA virus, belonging to the Reoviridae family (Day, 2009). The genome of MRVs is approximately 23.5 kb in length and contains 10 segments: three large (L1, L2 and L3), three medium (M1, M2 and M3) and four small (S1, S2, S3 and S4) segments, which encode eight structural proteins (λ1, λ2, λ3, μ1, μ2, σ1, σ2, σ3) and four nonstructural proteins (μNS, μNSC, σNS, σ1s) (Schiff, Nibert, and Tyler, 2007). The σ1 protein encoded by the S1 segment is responsible for cell-attachment, type-specific antisera neutralization and hemagglutinin activities. Based on the capacity of the 61 protein, MRVs have been divided into 4 serotypes: type 1 (MRV1) Lang (T1L), type 2 (MRV2) Jones (T2J), type 3 (MRV3) Dearing (T3D) and a putative 4 (MRV4) Ndelle (Attoui et al., 2001; Lelli et al., 2016).

MRVs are widely distributed and are able to infect many mammalian species including humans, pigs, bats, cattle, minks, dogs, cats and civets (Anbalagan, Spans, and Hause, 2014; Decaro et al., 2005; Kohl et al., 2012; Lelli et al., 2013; Li et al., 2015; Lian et al., 2013; Qin et al., 2017; Steyer et al., 2013; Yang et al., 2015). They are typically believed to cause mild gastroenteritis and respiratory disease which can be either symptomatic or asymptomatic in the infected species (Schiff, Nibert, and Tyler, 2007). MRVs have been identified in patients with acute respiratory infections and further shown to be potentially capable of human-to-human transmission in Malaysia (Chua et al., 2008; Chua et al., 2011). In Asian countries, especially in China and South Korea, the MRV3 virus has been responsible for some cases with severe neonatal diarrhea, fever and respiratory signs in pigs (Dai et al., 2012; Kwon et al., 2012; Zhang et al., 2011); MRV3 infections that resulted in diarrhea in pigs were also reported in Europe (Lelli et al., 2016). In 2015, a swine MRV3 was first reported in the U.S., which caused acute gastroenteritis, indicating that MRVs could be an important pathogen for the swine industry (Cao et al., 2018; Thimmasandra Narayanappa et al., 2015). Reoviruses have been considered to induce neurological symptoms even though fewer cases of those were reported in contrast to cases of enteric illness and respiratory signs. For example, necrotizing encephalopathy and meningitis induced by MRV2 or MRV3 in infected humans have been documented in Europe and the U.S. (Ouattara et al., 2011; Tyler et al., 2004). In addition, both MRV1 and MRV3 can infect the central nervous system (CNS) in mice by different pathways (Spriggs and Fields, 1982; Weiner et al., 1977). All available evidence indicates that MRVs are likely to be responsible for more severe diseases with exception to mild gastroenteritis and respiratory disease.

When multiple lineages of MRVs infect the same host, the segmented nature of MRVs results in reassortment to promote viral evolution and generate novel strains. Reassortant MRV strains have been identified in different species including vole, partridge, bat and calf (Anbalagan, Spaans, and Hause, 2014; Feher et al., 2017; Kugler et al., 2016; Lelli et al., 2015; Wang et al., 2015). Further studies showed that gene segments of these reassortant MRVs could be from different hosts based on sequence and phylogenetic analysis (Anbalagan, Spaans, and Hause, 2014; Feher et al., 2017; Kugler et al., 2016; Lelli et al., 2015; Wang et al., 2015). Additionally, a novel orthoreovirus detected in a hospitalized child with acute gastroenteritis showed high similarity to mammalian orthoreoviruses found in bats in Europe (Steyer et al., 2013). All facts indicate interspecies transmission of MRVs. In this study, we isolated a novel MRV strain from diseased pigs in a US Midwest swine farm in which more than 300 pigs showed neurological signs with approximately 40% mortality. Sequence and phylogenetic analysis revealed that the isolate was a reassortant virus, having the S1 segment from bovine-derived MRV1, the M2 segment from MRV2 and remaining eight segments from pig-derived MRV3. Further animal studies showed that the novel MRV isolate was able to infect and cause disease in pigs and transmitted to contact animals.

What is needed is an immunogenic composition that reduces the severity or incidence of clinical signs of orthoreovirus infection in a subject or group of subjects. What is also needed is an immunogenic composition that reduces the severity or incidence of clinical signs of orthoreovirus infection in a subject or group of subjects wherein the immunogenic composition includes at least one segment from a different strain of orthoreovirus and/or a different host of orthoreovirus than the other segments included in the composition. What is also needed is an immunogenic composition that reduces the severity or incidence of clinical signs of orthoreovirus infection in a subject or group of subjects wherein the immunogenic composition includes at least one segment from a different serotype of orthoreovirus than the other segments included in the composition.

### BRIEF DESCRIPTION OF THE DISCLOSURE

Mammalian orthoreovirus (MRV) is able to infect multiple mammalian species including humans. A U.S. Midwest swine farm with approximately one thousand 3-month-old pigs experienced an event, in which more than 300 pigs showed neurological signs, like “down and peddling”, with approximately 40% mortality. A novel MRV was isolated from the diseased pigs. Sequence and phylogenetic analysis revealed that the isolate was a reassortant virus containing viral gene segments from three MRV serotypes that infect human, bovine and swine. The M2 and S1 segment of the isolate showed 94% and 92% homology to that of the MRV2 D5/Jones and that of the MRV1 C/bovine/Indiana/MRV00304/2014, respectively; the remaining eight segments displayed 93%-94% homology to those of the MRV3 FS-03/Porcine/USA/2014. Pig studies showed that both MRV-infected and native contact pigs displayed fever, diarrhea and nasal discharge. MRV RNA was detected in different intestinal locations of both infected and contact pigs, indicating that the MRV isolate is pathogenic and transmissible in pigs. Seroconversion was also observed in infected pigs. A prevalence study on more than 200 swine serum samples collected from three states revealed 46-98% positive to MRV. All results warrant the necessity to monitor MRV epidemiology and reassortment as the MRV could be an important pathogen for the swine industry and a novel MRV might emerge to threaten animal and public health.

In one aspect, the present disclosure provides an immunogenic composition that includes at least one segment, or portion thereof, (“the different segment”) from a different serotype, host, and/or strain than the other segments included in the composition. In forms, the composition will include segments, or at least one portion thereof, from 1, 2, 3, or 4 different serotypes and/or at least 1, 2, 3, 4, 5, 6, 7, 8, 9, or even 10 different hosts, and/or strains. In some forms, the at least one different segment, or portion thereof, will be a large segment. In some forms, the at least one different segment, or portion thereof, will be a medium segment. In some forms, the at least one different segment, or portion thereof, will be a small segment. In some forms, the portion thereof will be a nucleotide sequence having at least 45, 48, 51, 54, 57, 60, 63, 66, 69, 72, 75, 78, 81, 84, 87, 90, 93, 96, 99, 102, 105, 108, 111, 114, 117, 120, 123, 126, 129, 132, 135, 138, 141, 144, 147, 150, 153, 156, 159, 162, 165, 168, 171, 174, 177, 180, 183, 186, 189, 192, 195, 198, 201, or more nucleotides. In some forms, the portion thereof will encode a structural protein. In some forms, the portion thereof will encode a nonstructural protein. In some forms, the structural protein will be selected from the group consisting of λ1, λ2, λ3, μ1, μ2, σ1, σ2, or σ3. In some forms, the nonstructural protein will be selected from the group consisting of μNS, μNSC, σNS, or σ1s. In some forms, the structural protein will be the σ1 protein. In some forms, the composition will include a total of 2, 3, 4, 5, 6, 7, 8, 9, 10, 12, 13, 14, 15, 16, 17, 18, 19, 20, 21, 22, or more segments, or portion(s) thereof. In some forms, the composition will include 1, 2, 3, or more large segments. In some forms, the composition will include 1, 2, 3 or more medium segments. In some forms, the composition will include 1, 2, 3, 4, or more small segments. In some forms, the small segment will include 1, 2, 3, 4, 5, 6, 7, 8, or more regions that code for a structural protein. In some forms, the small segment will include 1, 2, 3, 4, 5, 6, 7, 8, or more structural proteins. In some forms, the small segment will include 1, 2, 3, 4, or more regions that code for a nonstructural protein. In some forms, the small segment will include 1, 2, 3, 4, or more nonstructural proteins. In some forms, the composition will include more or fewer segment types, or portion(s) thereof than orthoreovirus. For example, the composition may include more than one 61 protein, more than 4 small segments, less than 4 small segments, more or less than 3 large segments, more or less than 3 medium segments, more or less than 8 structural proteins, more or less than 4 nonstructural proteins, and any potential combination of segment numbers. In some forms, the immunogenic composition will comprise the nucleotides of the segment(s), or portion(s) thereof set forth above. In some forms, the immunogenic composition will comprise a protein encoded by any of segment(s), or portion(s) thereof, set forth above. In some forms, the segment(s) or portions thereof will include a portion having at least 80, 81, 82, 83, 84, 85, 86, 87, 88, 89, 90, 91, 92, 93, 94, 95, 96, 97, 98, 99, 99.5, or even 100% sequence homology or identity to at least one of SEQ ID NOs. 4-13 or to the amino acid sequence encoded by at least one of SEQ ID NOs. 4-13. In some forms, the encoded amino acid sequence will have at least 80, 81, 82, 83, 84, 85, 86, 87, 88, 89, 90, 91, 92, 93, 94, 95, 96, 97, 98, 99, 99.5, or even 100% sequence homology or identity to at least one of SEQ ID NOs. 14-25. In some forms, an example of the λ1 portion is provided by SEQ ID NO. 14. In some forms, an example of the λ2 portion is provided by SEQ ID NO. 15. In some forms, an example of the λ3 is provided by SEQ ID NO. 16. In some forms, an example of the μ1 portion is provided by SEQ ID NO. 17. In some forms, an example of the μ2 portion is provided by SEQ ID NO. 18. In some forms, an example of the σ1 portion is provided by SEQ ID NO. 19. In some forms, an example of the σ2 portion is provided by SEQ ID NO. 20. In some forms, an example of the σ3 portion is provided by SEQ ID NO. 21. In some forms, an example of the μNS portion is provided by SEQ ID NO. 22. In some forms, an example of the μNSC portion is provided by SEQ ID NO. 23. In some forms, an example of the σNS portion is provided by SEQ ID NO. 24. In some forms, an example of the σ1s portion is provided by SEQ ID NO. 25.

In some forms, the assembled sequences will be live. In some forms, the assembled sequences will be inactivated. In some forms, the assembled sequences will be killed. In some forms, the immunogenic composition will comprise isolated and killed and/or inactivated whole mammalian orthoreovirus. Additional components, such as those noted herein may be added in effective amounts.

In some forms, the immunogenic composition will comprise only the segments or portions thereof that induce neutralizing antibodies or cellular mediated immunity. Additional components, such as those noted herein may be added in effective amounts.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

Those of skill in the art will understand that the composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others. Suitable adjuvants, are those described herein.

According to a further aspect, the immunogenic composition of the present disclosure further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

The immunogenic compositions described herein can further include one or more other immunomodulatory agents such as, e.g., interleukins, interferons, or other cytokines. The immunogenic compositions can also include Gentamicin and Merthiolate. In another preferred embodiment, the present disclosure contemplates vaccine compositions comprising from about 1 ug/ml to about 60 μg/ml of antibiotics, and more preferably less than about 30 μg/ml of antibiotics.

In some forms, the immunogenic composition of the disclosure includes an additional component selected from the group consisting of a stabilizer, an adjuvant, an antimicrobial, an antifungal, a preservative, an immunomodulatory agent, and any combination thereof.

In another aspect, the immunogenic composition described herein will be administered to an animal in need thereof in order to reduce the severity of or incidence of clinical signs of orthoreovirus infection. This includes both treatment of active orthoreovirus infection as well as the prevention of future orthoreovirus infection.

It is understood that “prevention” as used in the present disclosure, includes the complete prevention of infection by orthoreovirus, but also encompasses a reduction in the severity of or incidence of clinical signs associated with or caused by orthoreovirus infection. Such prevention is also referred to herein as a protective effect.

In one aspect, the composition(s) of the present disclosure reduces the clinical symptoms and/or the severity or incidence of clinical symptoms of orthoreovirus infection in a subject or group of subjects by at least 10%, at least 20%, at least 30%, at least 40%, at least 50%, at least 60%, at least 70%, at least 80%, at least 90%, at least 95%, from about 10% to 50%, from about 10% to 90%, from about 30% to 50%, from about 20% to 60%, from about 30% to 80%, from about 30% to 50%, and from about 50% to 95%, with all values within the recited ranges being envisioned. The reduction of clinical symptoms and/or the reduction of the incidence or severity of clinical symptoms is when compared to a subject or group of subjects not administered the immunogenic composition or vaccine of the present disclosure.

A subject or group of subjects is generally an animal and more particularly a mammal. Preferred subjects include swine and humans.

A method for preventing clinical symptoms or reducing the incidence or severity of symptoms of subclinical infection of orthoreovirus is also provided, where the steps of the method include administration of the immunogenic composition or vaccine against orthoreovirus to a subject in need thereof.

Subclinical infection presents with virus shedding and mild symptoms included, but not limited to, mild fever and/or loss of appetite. Clinical signs include subclinical signs with more severe fever, inappetance, weight loss, diarrhea, enteritis, nasal discharge, cough, encephalitis, and combinations thereof.

In one aspect, the method of the present invention reduces the symptoms of clinical and subclinical orthoreovirus infection and/or the severity or incidence of the symptoms of subclinical orthoreovirus infection in a subject or group of subjects, by at least 10%, at least 20%, at least 30%, at least 40%, at least 50%, at least 60%, at least 70%, at least 80%, at least 90%, at least 95%, from about 10% to 50%, from about 10% to 90%, from about 30% to 50%, from about 20% to 60%, from about 30% to 80%, from about 30% to 50%, and from about 50% to 95%, with all values within the recited ranges being envisioned. The reduction of clinical and subclinical symptoms and/or the reduction of the incidence or severity of clinical and subclinical symptoms is when compared to a subject or group of subjects not administered the immunogenic composition or vaccine of the present disclosure.

Compositions of the present disclosure can be administered by the systemic route. Alternatively, the composition of the present disclosure can be administered intranasally, orally, transdermally (i.e., applied on or at the skin surface for systemic absorption), parenterally, etc. The parenteral route of administration includes, but is not limited to, intramuscular, intravenous, intraperitoneal, subcutaneous, intradermal (i.e., injected or otherwise placed under the skin) routes and the like.

When administered as a liquid, the present vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

Their administration modes, dosages and optimum pharmaceutical forms can be determined according to the criteria generally taken into account in the establishment of a treatment adapted to a subject such as, for example, the age or the weight, the seriousness of its general condition, the tolerance to the treatment and the secondary effects noted. Preferably, the immunogenic composition of the present disclosure is administered in an amount that is protective or provides a protective effect against orthoreovirus infection.

For example, in the case of a vaccine or immunogenic composition according to the present disclosure comprising a nucleotide sequence, preferably a segment or portion thereof as described herein, of orthoreovirus, or an amino acid sequence encoded by such a nucleotide sequence or fragment thereof, the composition will be administered one time or several times, spread out over time, in an amount of about 0.1 μg to 1 g per kilogram weight of the subject for each dose.

In another aspect, a method of making the immunogenic composition described herein is provided. In one aspect that combines multiple segments, a cocktail strategy that includes multiple antigens (proteins) or DNAs with adjuvants is used to produce subunit vaccine (combination of multiple proteins) or DNA vaccine with several optimized segments. In other forms, the segments can be assembled using any conventional method including the mapping method.

In some forms, a recombinant viral vector containing at least one orthoreovirus nucleic acid segment is formed to express a desired amino acid sequence, such as those described herein. In some forms, such a viral vector containing desired orthoreovirus nucleic acid and encoding or expressing desired orthoreovirus amino acid sequences, such as those described herein, is used to infect cells by transfecting a transfer vector that has had an orthoreovirus segment, or portion thereof, cloned therein into a viral vector. Preferably, only the portion of the transfer vector that contains the desired orthoreovirus nucleic acid, such as the segments or portions thereof described herein, is transfected into the viral vector.

The term “transfected into a viral vector” means, and is used as a synonym for “introducing” or “cloning” a heterologous nucleic acid into a viral vector, such as for example into a baculovirus vector. A “transfer vector” means a nucleic acid molecule, that includes at least one origin of replication, the heterologous gene, in the present case of orthoreovirus, nucleic acid sequences which allow the cloning of said heterologous gene into the viral vector will be included. Preferably the sequences which allow cloning of the heterologous gene into the viral vector are flanking the heterologous gene. Even more preferably, those flanking sequences are at least homologous in parts with sequences of the viral vector. The sequence homology then allows recombination of both molecules, the viral vector, and the transfer vector to generate a recombinant viral vector containing the heterologous gene.

Additionally, the composition can include one or more pharmaceutical-acceptable or veterinary-acceptable carriers. As used herein, “a pharmaceutical-acceptable carrier” or “veterinary-acceptable carrier” includes any and all solvents, dispersion media, coatings, stabilizing agents, diluents, preservatives, antibacterial and antifungal agents, isotonic agents, adsorption delaying agents, and the like.

“Adjuvants” as used herein, can include aluminum hydroxide and aluminum phosphate, saponins e.g., Quil A, QS-21 (Cambridge Biotech Inc., Cambridge Mass.), GPI-0100 (Galenica Pharmaceuticals, Inc., Birmingham, Ala.), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). JohnWiley and Sons, NY, pp 51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta Ga.), SAF-M (Chiron, Emeryville Calif.), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

### BRIEF DESCRIPTION OF THE DRAWINGS

FIG. 1A is a phylogenetic tree from the 51 segment of novel reassortant MRV/USA/Porcine/2018;

FIG. 1B is a phylogenetic tree from the S2 segment of novel reassortant MRV/USA/Porcine/2018;

FIG. 1C is a phylogenetic tree from the S3 segment of novel reassortant MRV/USA/Porcine/2018;

FIG. 1D is a phylogenetic tree from the S4 segment of novel reassortant MRV/USA/Porcine/2018;

FIG. 1E is a phylogenetic tree from the M1 segment of novel reassortant MRV/USA/Porcine/2018;

FIG. 1F is a phylogenetic tree from the M2 segment of novel reassortant MRV/USA/Porcine/2018;

FIG. 1G is a phylogenetic tree from the M3 segment of novel reassortant MRV/USA/Porcine/2018;

FIG. 1H is a phylogenetic tree from the L1 segment of novel reassortant MRV/USA/Porcine/2018;

FIG. 1I is a phylogenetic tree from the L2 segment of novel reassortant MRV/USA/Porcine/2018;

FIG. 1J is a phylogenetic tree from the L3 segment of novel reassortant MRV/USA/Porcine/2018;

FIG. 2A is a photograph illustrating the detection of MRV antigens in infected MDCK cells by IFA;

FIG. 2B is a photograph illustrating the detection of MRV antigens in mock-infected cells;

FIG. 3A is a graph illustrating viral RNA detection through MRV RNA copy number in rectal swab samples collected from infected and control pigs;

FIG. 3B is a graph illustrating viral RNA detection through genomic MRV RNA copy number in nasal swab samples collected from infected and control pigs;

FIG. 4A is a photograph illustrating an analysis of sections of intestine and brain of infected and control pigs by H&E with prominent lymphoid follicular development in an infected pig wherein the scale bar is 500 μm;

FIG. 4B is a photograph illustrating an analysis of sections of intestine and brain of infected and control pigs by IHC for MRV with strong positive staining of follicular associated epithelium (FAE) and underlying lymphoid tissue in an infected pig. No staining was noted in lymphoid follicles proper wherein the scale bar is 500 μm;

FIG. 4C is a photograph representing segments of FAE overlying lymphoid follicles in the terminal ileum of in an infected pig. Staining was consistently present on the lower lateral aspect of the FAE with mild staining in the lamina propria between the epithelium and underlying lymphoid follicle proper (Scale bar, 50 μm);

FIG. 4D is a photograph of an image that represents segments of FAE overlying lymphoid follicles in the terminal ileum of in a control pig (Scale bar, 50 μm); and

FIG. 4E is a photograph illustrating an H & E stain of the section from base of cerebellum from inoculated pig #20. Prominent perivascular cuffs of lymphocytes, macrophage like cells, and rare plasma cells. There is mild diffuse gliosis. In other areas, distinct foci of gliosis are prominent (Scale bar, 100 μm).

### DETAILED DESCRIPTION OF THE INVENTION

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Materials and Methods

Ethics Statement

The animal experiment to investigate pathogenicity and transmissibility of the MRV isolate in pigs was reviewed and approved by the Institutional Animal Care and Use Committee at Kansas State University and was performed in Biosafety Level 2+ animal facilities under guidance from the Comparative Medicine Group at Kansas State University.

Pigs

Twenty-one 5-week-old pigs, which were seronegative to swine influenza virus and porcine respiratory and reproductive syndrome virus, were used in this study. We also confirmed that these pigs were seronegative to MRV by testing blood samples using hemagglutinin inhibition assay, and negative to porcine epidemic diarrhea virus, transmissible gastroenteritis Virus and porcine group A rotavirus by testing rectal swab samples collected from each pig using the specific real-time qPCR assays.

Clinical Case

In March 2018, a U.S. Midwest swine farm with approximately one 1,000 3-month-old pigs experienced a severe disease event, in which more than 300 pigs showed neurological signs “down and peddling” without diarrhea and 120 pigs died. The resident veterinarian euthanized and necropsied two diseased pigs, and the tissue samples including brain, kidney, spleen, lung, liver, heart, intestine and stomach fundus from each pig were collected and submitted to Kansas State Veterinary Diagnostic Laboratory for diagnosis.

Virus Isolation and Preparation

A homogenous mixture of brain, kidney, spleen, lung, liver, heart, intestine, and stomach fundus sample from each pig was made for routine diagnostics and virus isolation. Homogenized tissues were filtered and inoculated onto a monolayer of MDCK cells, which were grown in Minimal Essential Medium (MEM), supplemented with 3% bovine serum albumin (BSA) (Sigma-Aldrich), 1 μg/ml N-tosyl-L-phenylalanine chloromethyl ketone (TPCK)-treated trypsin (Sigma-Aldrich) and 1% antibiotic-antimycotic (Gibco) at 37° C. in the atmosphere with 5% CO₂. The inoculated cells were monitored for cytopathic effects (CPE) every 12 hours and were blind-passaged for three passages.

Cell culture supernatant was collected and centrifuged at 5,000 rpm for 10 minutes at 4° C. to remove cellular debris after clear CPE were observed. The centrifuged sample was then processed for ultra-centrifugation with 40% sucrose at 25,000 rpm at 4° C. for 2 hours. Both RNA and DNA were extracted from the ultra-centrifuged samples (QIAamp cador Pathogen Mini Kit, QIAGEN, USA) for target-specific PCR assays and next generation sequencing.

Pan-Viral Group PCR

Pan-viral family/genus PCRs and sequencing were performed for the following viral families/genera: Coronaviridae, Herpesviridae, Orthomyxoviridae (Influenza viruses A, B and C), and Reoviridae (Aquareovirus and Orthoreovirus) (Phaneuf et al., 2013; Tong et al., 2009). First round reverse transcription PCR for RNA viruses was performed with Superscript III/Platinum Taq One Step kits (Invitrogen) and Titanium Taq (Clontech) kits for the second round PCR. First and second round PCR for DNA viruses was performed with Hot Start Ex Taq kits (Takara). A positive PCR control containing mutation-engineered synthetic RNA transcript or DNA amplicon and a negative control using nuclease-free water were included in each run. PCR products were visualized on 2% agarose gels. Positive bands of the expected size that had strong signal and without additional bands were purified using Exonuclease I (New England Biolabs) and Shrimp Alkaline Phosphatase (Roche). Samples were incubated at 37° C. for 15 minutes followed by 80° C. for 15 minutes to inactivate the Exonuclease and Shrimp Alkaline Phosphatase. Purified PCR amplicons were sequenced with the PCR primers in both directions on an ABI Prism 3130 Automated Capillary Sequencer (Applied Biosystems) using Big Dye 3.1 cycle sequencing kits (Life Technologies).

Next Generation Sequencing (NGS) and Analysis

Extracted nucleic acids (NA) were pre-amplified using a modified random amplification protocol as described previously (Tong et al., 2012). Briefly, NA samples are reverse-transcribed using a primer containing both a known sequence and a random nanomer, followed by a second primer extension reaction using the same primer. These extension fragments are then amplified by PCR using the known sequence of the extension primer. PCR amplicons obtained from the pre-amplification were purified, fragmented and used to construct libraries with dual index barcoding for Illumina sequencing on a MiSeq instrument as previously described (Li et al., 2017).

Initially, reads were assembled and classified using SURPI (Naccache et al., 2014). Subsequently, reovirus reads identified by SURPI were extracted and further analyzed by de novo assembly as well reference-based assembly with Geneious v11.1.4. Consensus sequences of all genomic segments were generated by Geneious and used for phylogenetic analysis.

Sequence and Phylogenetic Analysis

Each segment sequence of the isolate was blasted and compared with available sequences in GenBank, and the hit with the best identity for each gene was recorded. Each segment sequences of the closely related and other typical reovirus strains were downloaded for further phylogenetic analysis. Maximum likelihood phylogenetic tree of each segment based on the open reading frame was built with MEGA 5.0 using the Jukes-Cantor model with a bootstrap value of 1,000. GenBank accession numbers for each segment of this isolate are pending.

Immunofluorescence (IFA) Assay

IFA assay was employed to confirm the MRV isolate using a mouse monoclonal antibody against reovirus capsid protein μ1C (10F6; DSHB, USA). A monolayer of MDCK cells was infected with the isolated MRV for 48 hours, and then fixed with 10% methanol for 10 minutes following 3 washes using phosphate buffered saline with tween 20 (PBST). After blocking with 5% fetal bovine serum (FBS) for 1 hour at room temperature and washing cells with PBST for 3 times, the cell monolayer was incubated with anti-reovirus capsid protein μ1C monoclonal antibody with 1:50 dilution at room temperature for 2 hours. The cell monolayer was then washed for 3 times and incubated with the second antibody FITC-labeled goat anti-mouse IgG (H+L) (Jackson ImmunoResearch, USA) with 1:200 dilution for 2 hours. After washing the cell monolayer for 3 times with PBST, fluorescence signals was observed under the microscope. Uninfected cells were processed in parallel and used as negative controls.

MRV Real-Time RT-PCR Assay

In order to detect all serotypes of MRVs, a quantitative real-time PCR (RT-qPCR_L1) was developed by targeting the conserved region of MRV L1 genes based on sequence information available in GenBank. The probe was labeled with 6-carboxyfluorescein and with 6-carboxytetramethylrhodamine at the 5′ and 3′ ends, respectively; and nucleotide information of primers and probe is summarized in Table 1. The RT-qPCR_L1 assay was performed by using qScript XLT 1-Step RT-qPCR ToughMix (Quantabio, Beverly, Mass., USA) according to the manufacturer's instructions with a total reaction system of 10 μl, which contained 5 μl of ToughMix, 0.48 μl of L1-F (12 μM), 0.48 μl of L1-R (12 μM), 0.16 μl of L1-Probe (4 μM), 1.38 μl of H₂O, and 2.5 μl of RNA template. The thermos-cycling conditions were set as follows: 50° C. for 10 min, 95° C. for 1 min, then 45 cycles of at 95° C. for 10 seconds and 60° C. for 45 seconds. The analytic sensitivity of the RT-qPCR_L1 assay was assessed using 10-fold serially diluted in vitro-transcribed RNA of the MRV isolate L1, gene ranging from 1×10⁻² to 1×10⁹ RNA molecules by two independent experiments according to above conditions.

TABLE 1
Primers and probe used in real-time RT-PCR assay

| | | Location of | Amplicon |
|---|---|---|---|
| | Primers/probe sequence | primers | (bp) |
| L1- | 5′-TGGCAGCGDTGGATACGTTATTC-3′ | 2521-2543 | 137 bp |
| probe | (SEQ ID NO. 1) | | |
| L1-F | 5′-GCGAAYTCTTCAGCRGAGGAGC-3′ | 2434-2455 | |
| | (SEQ ID NO. 2) | | |
| L1-R | 5′-CGTGARAAAGCACAGCATARAGCC-3′ | 2547-2570 | |
| | (SEQ ID NO. 3) | | |

Pig Study Design

Twenty-one 5-week-old pigs were randomly divided into 2 groups including one infected and one control group. Nine pigs were inoculated with the MRV isolate (1.0×10⁷ TCID₅₀/pig in 3 ml) intranasally (1.5 ml through the intranasal route) and orally (1.5 ml through the oral route), and three naïve pigs were commingled with infected pigs at 2 days post infection (dpi) to investigate virus transmission. Another nine pigs were mock-inoculated controls. Clinical signs and body temperature were monitored daily. Rectal swab sample was collected from each pig daily and nasal swabs were collected from each pig every two days. Blood samples were collected from each infected pigs at 0, 3, 5, 7, 9 and 14 dpi and from contact pigs at 0, 3, 5 and 7 days post contact (dpc). Three infected and control pigs were necropsied at 4 and 7 dpi, three contact pigs were necropsied at 7 dpc. During necropsy, tissue lesions was evaluated by an experienced pathologist; the tissues including duodenum, jejunum, ileum, colon, brain stem, lung, kidney, heart, liver and spleen were collected for further virological and histopathological analysis. The remaining three infected and control pigs were kept for 14 days to determine seroconversion.

Histopathology and Immunohistochemistry (IHC) Analysis

Tissues from brain, lung, liver, kidney, spleen, pancreas, stomach duodenum, jejunum, ileum ileocolic junction, spiral colon and descending colon collected from each pig were fixed in 10% neutral buffered formalin for further histopathological analysis. Based on initial PCR screening and clinical signs, a subset of positive tissues from infected pigs (n=3) along with matching tissues from the control animal at 4 dpi were routinely processed, and stained with hematoxylin and eosin and IHC. A board-certified veterinary pathologist evaluated histopathological lesions of each slide of stained tissues in a blinded fashion. IHC was conducted to detect MRV antigens by using the anti-reovirus capsid protein μ1C monoclonal antibody (1:80 diluted) and the second antibody Power-Vision Poly-AP anti-mouse IgG.

Hemagglutination Inhibition (HI) Assay

A total of 207 swine serum samples were collected from three states including Kansas, Texas and Minnesota. Serum sample collected from gnotobiotic, caesarian derived colostrum deprived piglets, which are MRV negative, was used for the negative control. All serum samples were treated by receptor destroying enzyme (RDE II) (DENKA SEIKEN, Japan) and adsorbed by using swine red blood cells (RBCs) prior to the HI assay. Briefly, serum samples were added into RDE II solution in the ratio of 1:3, and mixed thoroughly and incubated for 18 to 20 hours. After incubation, six volumes of 0.85% saline were added into the mixture and heated at 56° C. for 30 to 60 minutes to deactivate the RDE II. For each serum sample, 200 μl RDE II-treated serum was transferred to 96-well microtiter plates and 10 μl of 25% (v/v) swine RBCs was added into each well. After a gentle vortex, the plate was put at 4° C. for 1 hour and then centrifuged at 400×g for 10 min. The final 1:10 diluted serum samples were transferred to new 96 well plates. Four HA units of MRVs were used in the HI assay and added into serially diluted serum samples. The plates were gently mixed and incubated at room temperature for 1 hour, then 50 μl of 1% swine RBCs was added to each well and incubated at room temperature for another 1 hour. The HI titer for each sample was then determined.

Results

Initial Screening of Clinical Samples from Pigs with Neurological Signs

After receiving the tissue samples from two diseased pigs with neurological signs, the Kansas State Veterinary Diagnostic Laboratory performed a panel of routine molecular diagnosis, aerobic culture and histopathological analysis. Molecular diagnosis of tissue homogenate mixtures using RT-PCR or RT-qPCR assays showed that both porcine reproductive and respiratory syndrome virus and atypical porcine pestivirus were negative. Results of aerobic culture showed that Bordetella bronchiseptica was detected in lungs of one pig and Staphylococcus aureus was found in the brain of one pig after culturing for five days. Histopathological analysis revealed both pigs had moderate lymphohistiocytic, interstitial pneumonia in the lungs, moderate multifocal atrocytic hypertrophy and swelling with minimal gliosis in the brain and mild, lymphocytic, plasmacytic and eosinophilic enterocolitis in small intestine.

We used the tissue homogenates to test influenza A, B, C and D viruses, porcine teschovirus, sapelovirus and encephalomyocarditis virus by RT-PCR or real-time PCR assays. Results of these assays were all negative to tested pathogens. Virus isolation was performed on MDCK cells, and obvious CPE were observed at 48 hours post-infection at the third passages of the tissue homogenates from both pigs. The supernatant collected from infected cells showing CPE was tested above pathogens again, and results were negative to all these pathogens tested.

Isolation and Characterization of a Novel Reassortant MRV

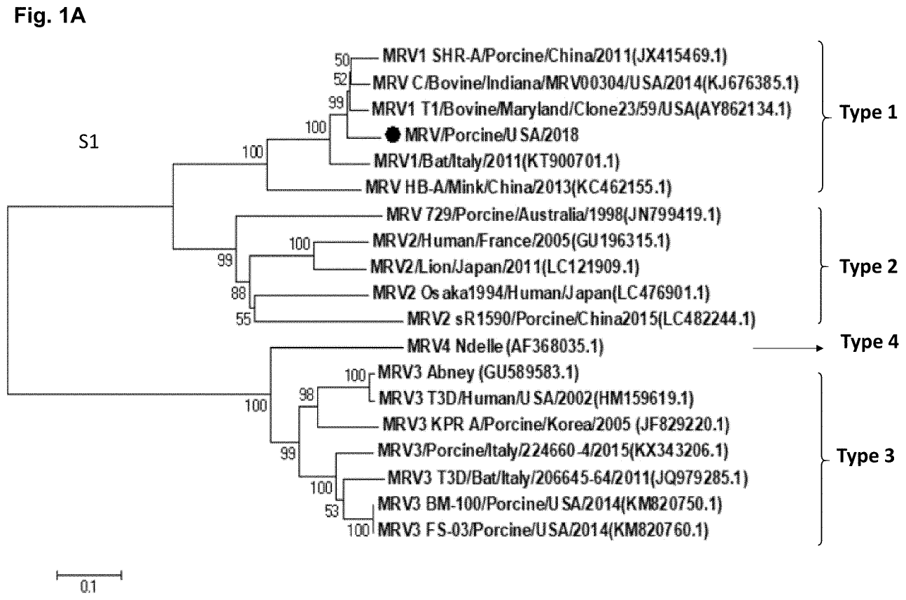

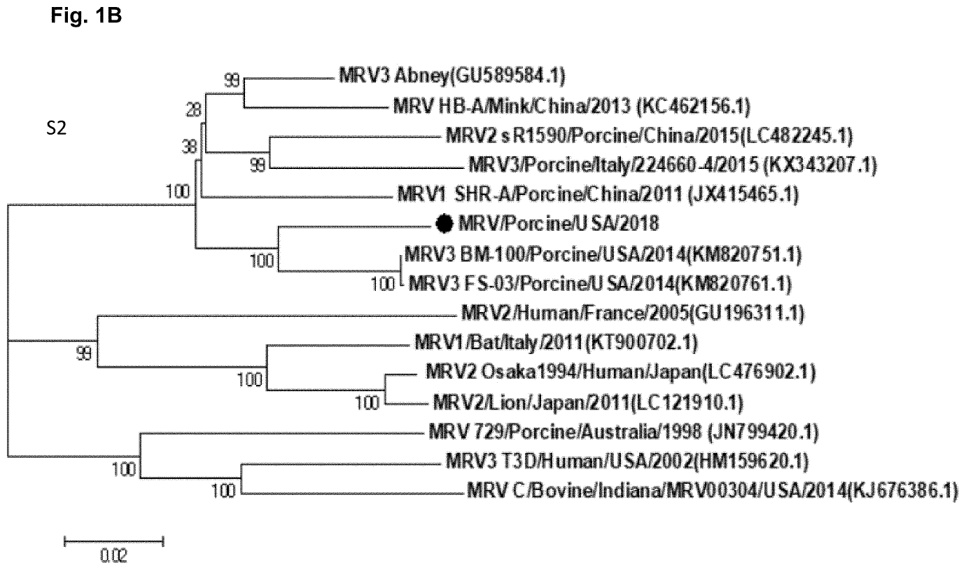

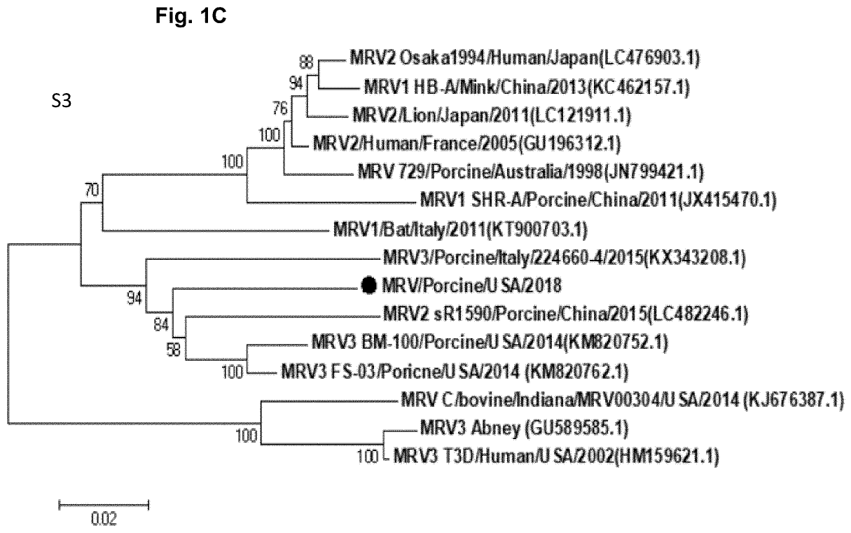

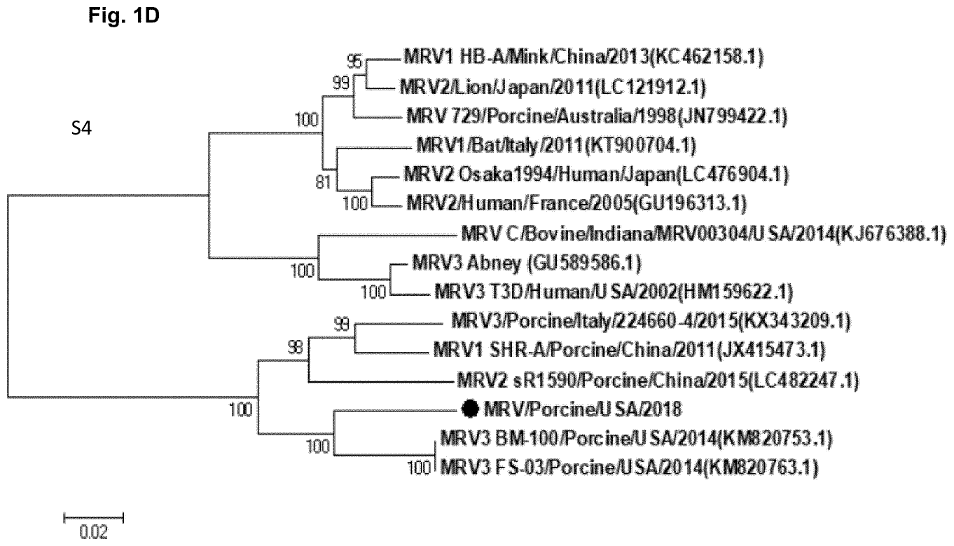

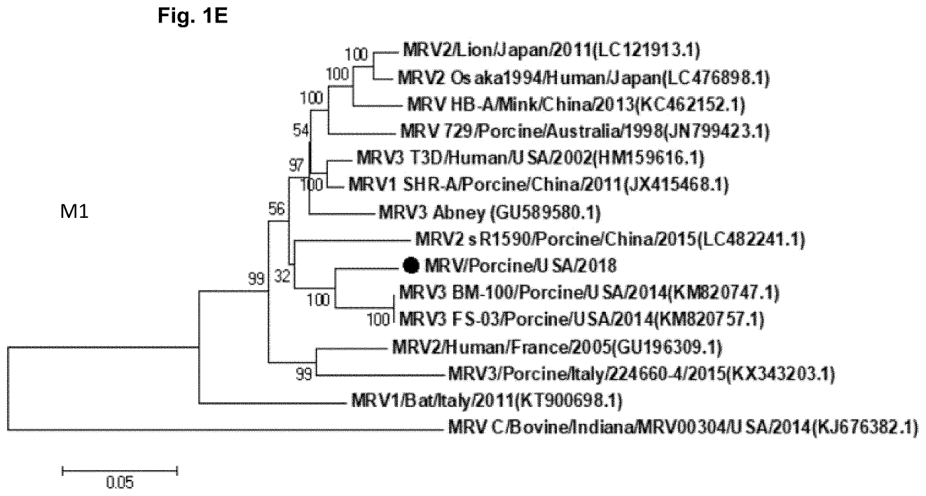

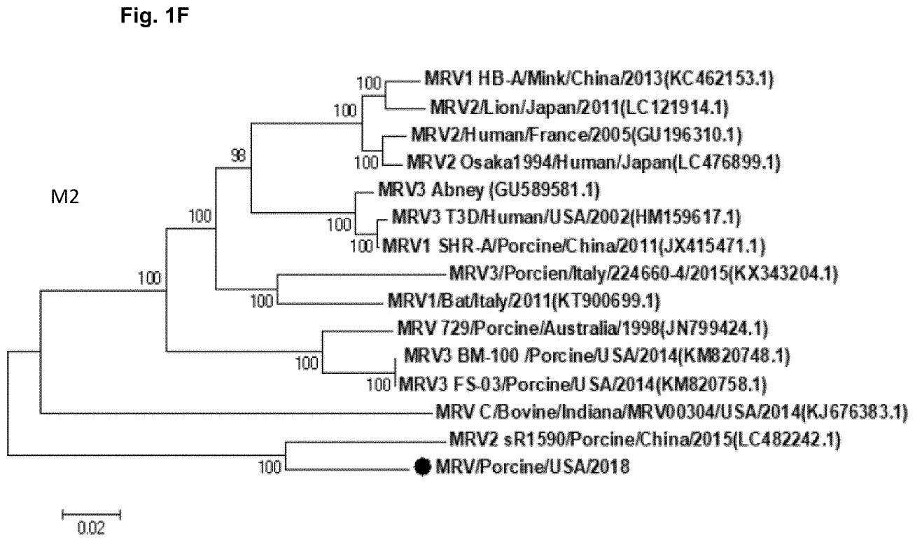

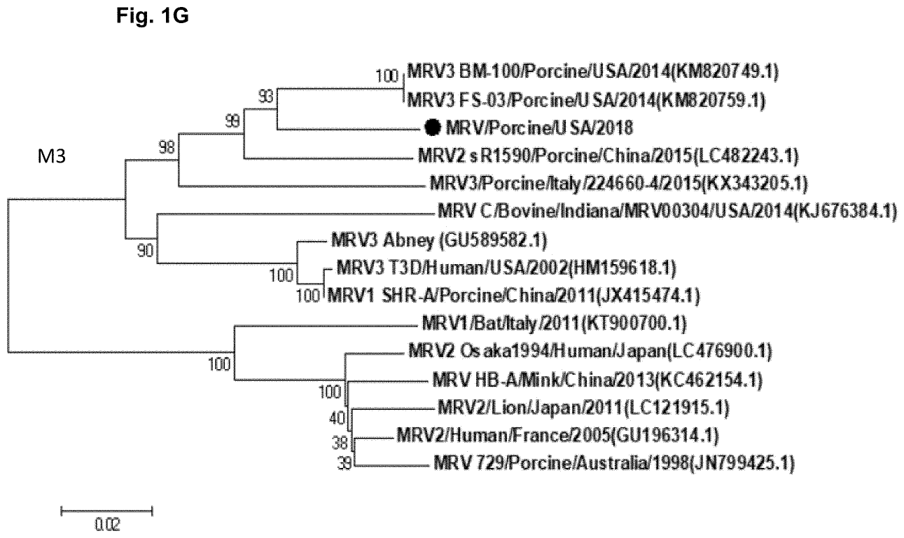

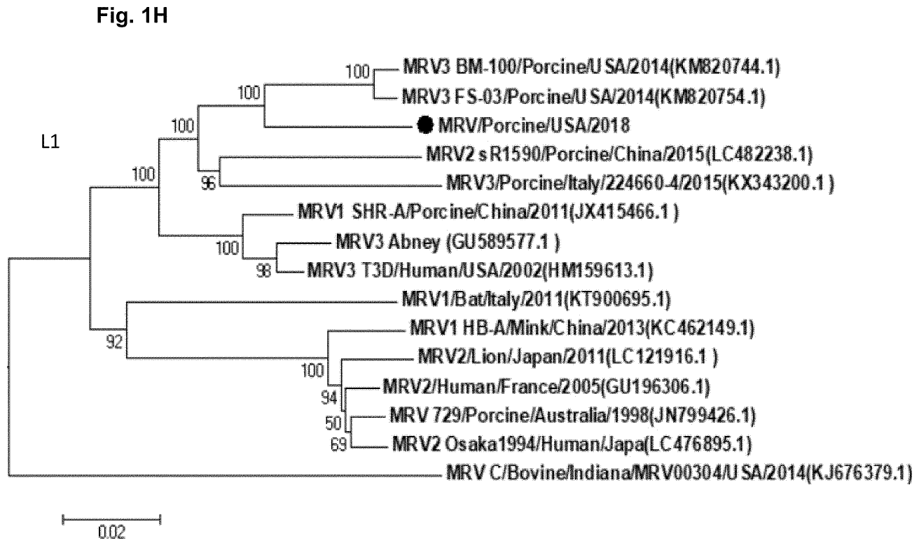

Both RNA and DNA were extracted from the ultra-centrifuged supernatants collected from infected MDCK cells displaying CPE for further testing. Pan-viral group PCR for Reoviridae was positive to mammalian orthoreovirus and negative to other viral families tested. Results of NGS revealed a novel MRV strain present in the sample, and full genome sequences of all 10 segments were obtained. Sequence analysis showed that the 51 segment displays 92% homology with a bovine-derived MRV1 (C/bovine/Indiana/MRV00304/2014) detected in bovine calves in the U.S.A. in 2014 (Anbalagan, Spaans, and Hause, 2014), the M2 segment is closely related to the human D5/Jones MRV2 strain (94% homology), while the remaining eight segments are highly homologous to the swine-origin MRV3 (T3/Swine/FS03/USA/2015 and T3/Swine/BM100/USA/2015) detected in pigs in the U.S.A. (Thimmasandra Narayanappa et al., 2015). The highest nucleotide homology for each segment of the isolate compared to available sequences of MRV strains in GenBank were depicted in Table 2. Phylogenetic tree of each segment of the virus was generated and shown in FIGS. 1A-J. Results of sequence and phylogenic trees indicate that the novel MRV isolate was a novel reassortant among three MRV serotypes (MRV1-3) and was named as the MRV/Porcine/USA/2018 (Table 2 and FIGS. 1A-J). Related MRV strains were downloaded from GenBank, and open reading fame of each gene segment was used for building the phylogenetic trees. In the figures, the MRV isolate identified in this study is labeled with a round dot.

TABLE 2
Highest nucleotide identities of MRV strains with each gene segment of
the novel reassortant MRV/Porcine/USA/2018.

| MRV/porcine/ | ldentity | MRV | | | GenBank |
|---|---|---|---|---|---|
| USA/2018 | % | strain | Serotype | Host | No. |
| L1 | 94 | FS-03/Porcine/USA/2014 | 3 | Pig | KM820754.1 |
| | 94 | BM-100/Porcine/USA/2014 | 3 | Pig | KM820744.1 |
| L2 | 94 | T3/Bovine/Maryland/1961 | 3 | Bovine | AF378008.1 |
| | 91 | FS-03/Porcine/USA/2014 | 3 | Pig | KM820755.1 |
| L3 | 95 | FS-03/Porcine/USA/2014 | 3 | Pig | KM820756.1 |
| | 95 | BM-100/Porcine/USA/2014 | 3 | Pig | KM820746.1 |
| M1 | 95 | FS-03/Porcine/USA/2014 | 3 | Pig | KM820757.1 |
| | 95 | BM-100/Porcine/USA/2014 | 3 | Pig | KM820747.1 |
| M2 | 94 | MRV2 D5/Jones | 2 | Human | M19355.1 |
| | 91 | MRV2 sR1590 | 2 | Pig | LC482242.1 |
| M3 | 94 | FS-03/Porcine/USA/2014 | 3 | Pig | KM820759.1 |
| | 94 | BM-100/Porcine/USA/2014 | 3 | Pig | KM820749.1 |
| S1 | 92 | C/bovine/Indiana/MRV00304/2014 | 1 | Bovine | KJ676385.1 |
| | 92 | T1/bovine/Maryland/Clone23/59 | 1 | Bovine | AY862134.1 |
| S2 | 95 | BM-100/Porcine/USA/2014 | 3 | Pig | KM820751.1 |
| | 94 | FS-03/Porcine/USA/2014 | 3 | Pig | KM820761.1 |
| S3 | 94 | FS-03/Porcine/USA/2014 | 3 | Pig | KM820762.1 |
| | 93 | BM-100/Porcine/USA/2014 | 3 | Pig | KM820752.1 |
| S4 | 93 | FS-03/Porcine/USA/2014 | 3 | Pig | KM820763.1 |
| | 93 | BM-100/Porcine/USA/2014 | 3 | Pig | KM820753.1 |

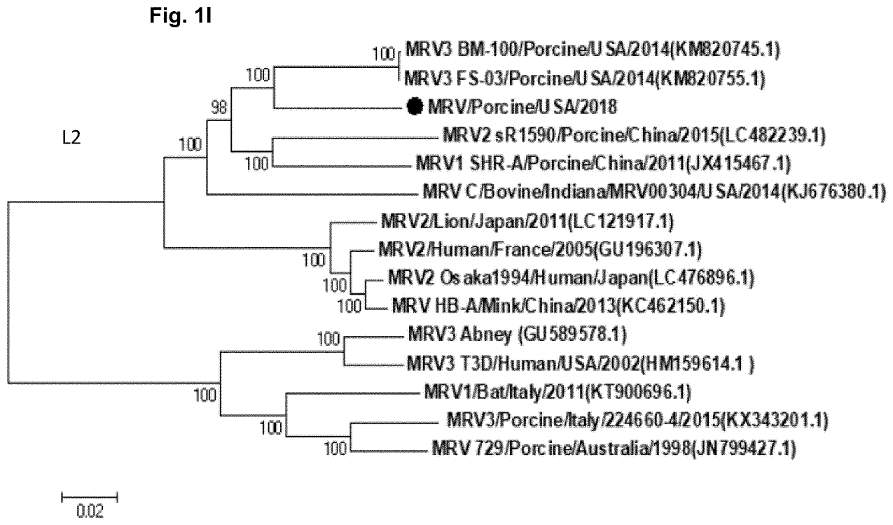

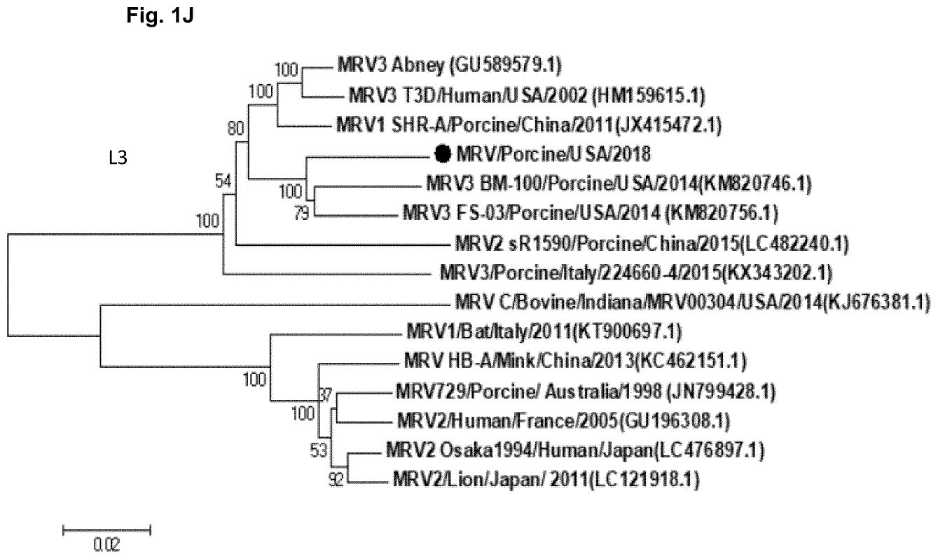

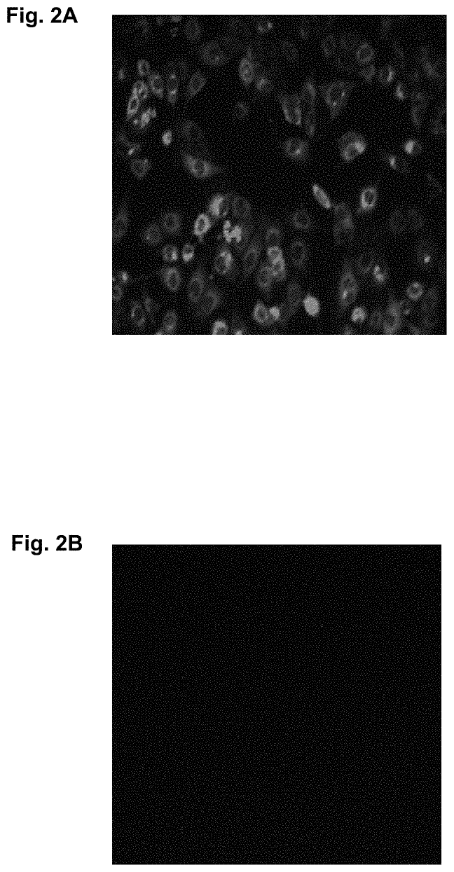

The MRV isolate was able to replicate efficiently and its titer could reach was 8 log₁₀ TCID₅₀ per mL in MDCK cells supplementing with TPCK-trypsin (1 μg/mL). The MRV antigen was mainly detected in the cytoplasm of infected MDCK cells by using an anti-reovirus μ1C protein monoclonal antibody 10F6 (FIGS. 2A and 2B). In FIGS. 2A and 2B, MDCK cells were mock-infected or infected with the novel MRV isolate for 48 hours and fixed for IFA assay. The fixed cells were incubated with the anti-mu 1C monoclonal antibody and the FITC-labeled goat anti-mouse IgG second antibody.

Sensitivity of Developed MRV Real-Time PCR

A real-time PCR (RT-qPCR_L1) was developed by targeting the conserved region of MRV L1 gene in order to quantify virus loads in samples collected from the pig study. To determine the sensitivity of the developed RT-qPCR_L1 assay, the L1 gene of the MRV isolate was cloned into a T7 promoter vector and in vitro transcribed to produce viral RNA. The analytic sensitivity of RT-qPCR_L1 assay showed a detection limit of 10 RNA copies and the cutoff of threshold cycle (CT) of this assay was 40. Further analysis showed a linear correlation of a series of RNA dilutions with R² higher than 0.999, indicating that this assay is reproducible and quantitative. We also detected RNA samples extracted from serially diluted MRV isolate with a known titer, and results showed that the assay was able to detect 100 TCID₅₀ per mL of the MRV virus. These results indicate that the developed RT-qPCR_L1 can be used to determine virus titer of samples collected in following pig studies.

Pathogenicity and Transmissibility of MRV in Pigs

Pigs in control group didn't show clinical signs through the length of the study. Clinical signs including fever, diarrhea and nasal discharge were observed in both infected and contact pigs. Six out of 9 infected pigs displayed fever (over 104° F.) starting at 2, 3 or 4 dpi, lasting for 2 to 3 days, and 7 out of 9 infected pigs showed diarrhea starting at 1 or 4 dpi, lasting for 1 to 4 days. Interestingly, 1 out of 9 infected pigs showed “walking discordant” (a neurological sign), starting at 2 dpi and lasting for 3 days, and 2 out of 9 infected pigs showed nasal discharge, starting at 6 dpi and lasting for 2 days. Noticeably, 2 out of 3 contact pigs developed fever, starting at 1 or 5 dpc and lasting for 1 day, and 2 out of 3 contact pigs had diarrhea, starting at 3 or 6 dpi and lasting for 2 to 3 days; 1 out of 3 showed nasal discharge, starting at 4 dpc and lasting for 2 days.

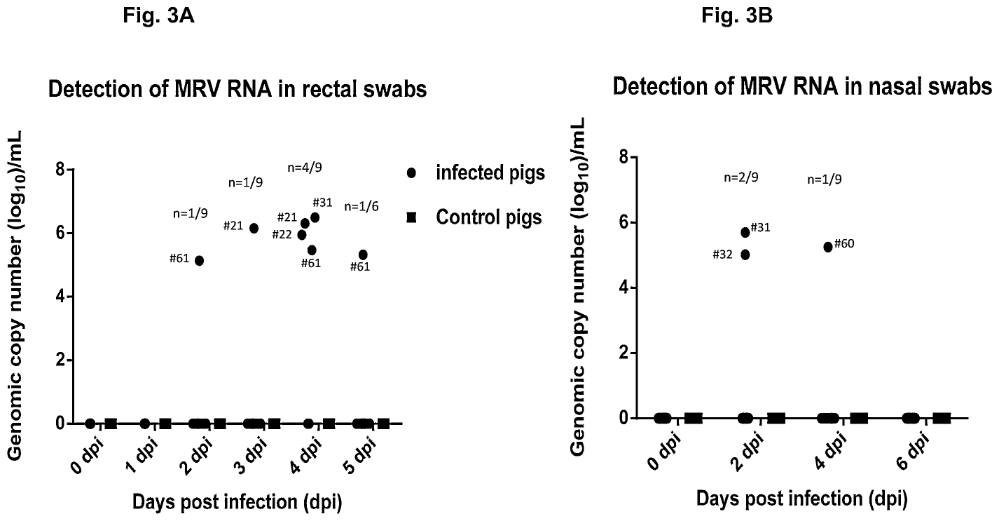

We employed the develop RT-qPCR_L1 to determine virus loads in collected samples during the pig study. No MRV viral RNA was detected in both nasal and rectal swab samples collected from control or contact pigs. MRV viral RNA was detected in rectal swab samples collected from one infected pig at 2, 4 and 5 dpi, and from one infected pig at 3 and 4 dpi and from another 2 infected pigs at 4 dpi (FIG. 3). In contrast, MRV viral RNA was detected in nasal swabs from two infected pigs at 2 dpi and from another infected pig at 4 dpi. Interestingly, rectal and nasal swab samples positive to MRV was only found in one infected pig.

MRV Viral RNA was only detected in the intestine, not in other tissues collected from both infected and contact pigs. Two infected pigs necropsied at 4 dpi and 2 contact pigs necropsied at 7 dpc showed MRV viral RNA positive in their intestine tissues. Viral RNA of 10^{7.69} molecules (per gram) was detected in the duodenum sample from one infected pig whose nasal swabs were positive to MRV at 4 dpi, while the intestine samples including duodenum, colon and ileum collected from another infected pigs were positive to MRV with a titer of 10^{5.20}. 10^{6.80} and 10^{7.93} RNA molecules per gram. In contrast, viral RNA of 10^{5.32} molecules (per gram) was detected in the colon sample of one contact pig, while the duodenum sample from another contact pig were positive to MRV with a titer of a titer of 10^{7.82} RNA molecules per gram. All infected pigs seroconverted at 7 dpi with HI titer ranging from 40 to 320 (the geometric mean was 71), and the HI titer increased at 9 and 14 dpi (the geometric mean was 101 and 202 at 9 and 14 dpi, respectively). However, no HI titer was detected in contact and control pigs.

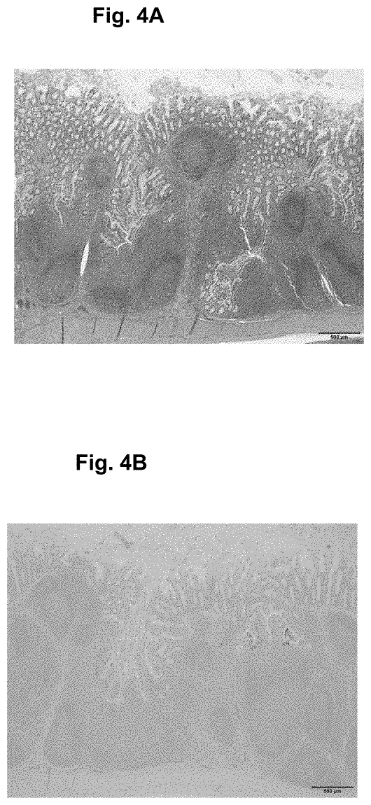

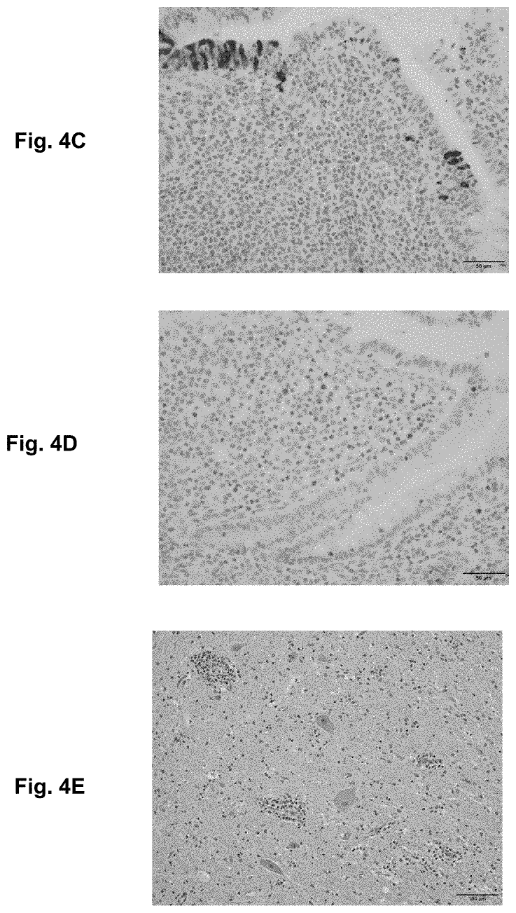

We found that 2 of 3 infected pigs which were necropsied at 4 and 7 respectively, had large and swollen mesenteric lymph nodes in contrast to control pigs. Other organs had no obvious changes in infected pigs compared to those of control pigs. Based on the positive results of initial PCR screening and clinical signs, the intestine sections from duodenum, mid jejunum and ileum and ileocecocolic junction, and bilateral sections of rostral cerebrum, thalamus, hippocampus, midbrain (colliculi), brainstem (obex, cerebral peduncles) and cerebellum from 3 infected pigs necropsied at 4 dpi were further examined. All intestine sections of 3 infected pigs had missing or exfoliating villous tip epithelium, and mild to moderate populations of lymphocytes and plasma cells and multifocally neutrophils in lamina propria (FIG. 4A). However, no significant difference was observed when compared to intestinal sections from the control pigs. IHC staining showed that 1 of 3 infected pigs had strong staining of follicle associated epithelium (FAE) within the ileum accompanied by mild to moderate staining of the underlying lymphocytes in lamina propria. There was no staining of epithelium away from ileal Peyer's patch or in adjacent lymph nodes (FIGS. 4B, C & D).

In sections of brain, one inoculated animal (pig #20) with clinical neurological sign described as “walking discordant”, had mild to moderate, multifocal, perivascular cuffs of lymphocytes, rare plasma cells, and macrophages along with scattered minimal to moderate collections of irregular glial cells (gliosis) throughout the thalamus, midbrain, and base of cerebellum (FIG. 4E). However, the results of the IHC staining were negative for any sections of brain of this pig and other 2 infected animals.

Sero-Prevalence of MRV in Pigs with Different Ages

To determine prevalence of MRV in pigs, a total of 228 serum samples were collected from pigs at Kansas, Texas, and Minnesota and tested by the HI assay using the novel isolate MRV/Porcine/USA/2018. These samples included 110 serum samples collected in 2018 from pigs at Minnesota and 70 samples collected in 2014 from pigs at Texas, which were approximately three-week-old; while the 47 samples collected in 2018 from pigs at Kansas which were approximately 3-month-old. The Kansas swine farm had experienced an outbreak of neurological disease while no obvious clinical signs was observed in pigs from both Texas and Minnesota. The 98% of serum samples from Kansas were positive to MRV, while serum samples from Texas and Minnesota displayed 46% and 63% positive to this virus, respectively. This results suggest the MRV is likely widespread in swine herds.

TABLE 3
MRV sero-prevalence in pigs at different geographical regions in the U.S.

| | | No. of | | | | |
|---|---|---|---|---|---|---|
| | No. of | MRV- | Positive | | Geometric | Collection |
| | samples | positive | Percent | HI titer | Mean | Time |
| Kansas | 47 | 46 | 98% | 10-320 | 50.1 | 2018 |
| Minnesota | 111 | 70 | 63% | 10-160 | 47.8 | 2018 |
| Texas | 70 | 32 | 46% | 10-80 | 24.3 | 2014 |

Discussion

In this study, we isolated a novel MRV/Porcine/USA/2018 from pigs in US Midwest swine farm in which approximately 300 pigs displayed neurological signs with approximately 40% mortality. Sequence and phylogenetic analysis revealed that this isolate was a reassortant strain with the 51 gene from a bovine-derived ressortant MRV1 (Anbalagan, Spaans, and Hause, 2014) and the M2 gene close to the human MRV2 D/Jones and the remaining eight genes from a swine-derived MRV3 that caused diarrhea in piglets in the U.S. in 2015 (Thimmasandra Narayanappa et al., 2015). Like influenza A viruses, the segmented nature of MRVs leads to reassortment among different serotypes or strains to produce novel viral strains. One former study shows that a novel reassortant bat MRV virus has the 51 gene similar to those from the bovine-derived MRV1 viruses and other remaining genes from bat MRV viruses (Lelli et al., 2015) and another study reveals that MRV isolates in different bat species in China are closely related to human, swine and mink orthoreoviruses (Yang et al., 2015). The novel reassortant MRV isolate reported in this study provides further evidence on reassortment among three MRV serotypes. However, how this novel virus was generated and whether an intermediates host was needed remains unknown and needs to be investigated. Importantly, bat-origin orthoreoviruses such as Melaka virus and Kampar virus have been associated with human infections (Chua et al., 2007; Chua et al., 2008; Chua et al., 2011). The MRV3 has been recorded to infect human and multiple animal hosts, and was recently identified in alpine chamois (Besozzi et al., 2019). In addition, a novel orthoreovirus that results in acute gastroenteritis in a hospitalized child has been isolated in Europe and revealed that the virus most likely originates from bats (Steyer et al., 2013). All these results demonstrate interspecies transmission and frequent reassortment events of MRVs.

Our pig studies indicate that the MRV isolate is pathogenic and transmissible in pigs, evidenced by disease, and virus replication and transmission found in both infected and contact pigs. Noticeably, only one of 9 infected pigs showed a neurological sign “walking discordant”, while most infected and contact pigs had diarrhea. Encephalitis was histologically observed in the brain of this pig although MRV antigens were not detected. In contrast, MRV antigens were detected in the FAE in the Peyers Patch of the ileum of infected pigs with diarrhea, which corresponds to the reported M cell distribution in pigs (Kido et al., 2003) and staining patterns seen in mouse reovirus experiments (Amerongen et al., 1994), suggesting the MRV adheres to the same cells to start replication in different species. The results of the pig study are not consistent with high mortality and neurological disease observed in diseased pigs in the outbreak farm. In addition, viral RNA and antigens were only detected in the intestine rather than brain of infected pigs in our study. This discrepancy suggests that other factors might be needed in order to reproduce the disease, such as unknown pathogens, environmental factor and stress etc. as cofactors. Thus, further studies are needed to understand what kind of co-factors are required to reproduce the neurological disease observed in pigs.

Encephalitis and meningitis caused by MRV infection has been documented in infected humans (Ouattara et al., 2011; Tyler et al., 2004). The pathway of MRV infection of CNS in newborn mice has been systemically investigated. Serotype 1 reovirus spreads to CNS via the hematogenous route, resulting in self-limiting hydrocephalus, while serotype 3 strain enters CNS by neutral routes and causes lethal encephalitis (Tyler, McPhee, and Fields, 1986; Weiner et al., 1977). Further studies to investigate dissemination pathways and neural tropism using serotype 1 and 3 reassortant clones suggest that reovirus virulence to CNS may be related to specific interactions between hemagglutinin and neuronal surface receptor (Dichter and Weiner, 1984; Tardieu and Weiner, 1982; Weiner, Powers, and Fields, 1980). Based on our knowledge, the novel MRV strain described in this study is the second isolate associated with neurological disease in pigs. The first MRV related to neurological disease is a MRV2 virus which was isolated from a pig with encephalitis in Austria. In contrast to two former swine MRV3 stains identified in the U.S. in 2015 that caused diarrhea in piglets (Thimmasandra Narayanappa et al., 2015), the novel MRV isolate has a different 51 and M2 gene, suggesting that they might be responsible for inducing different phenotypes of disease in pigs. Further studies are needed to investigate their roles in virus pathogenicity and tissue tropism using reverse genetics.

We have shown a high MRV sero-prevalence in pigs in the USA at different ages, suggesting the MRV could be widespread and an important pathogen for swine industry. In humans, there is a very high MRV sero-prevalence in infants, likely related to maternal antibody because seropositive rates can be up to 75% in 0-3 month old infants and 11% in 3-6 month old babies, while it decreases to 0% in children at 6-12 months of age (Tai et al., 2005). Approximately 50% sero-positivity in post-weaned (3-week-old) piglets at two farms located in different states suggests that the MRV-seropositive could also be associated with maternal antibodies. However, 3 to 6-month-old pigs in the disease outbreak farm were 98% MRV-seropositive, indicating that MRV is capable of causing severe infections in pigs. Additionally, previous studies have revealed that MRV3 σ1-based indirect ELISA assay can also detect MRV serotype 1 strains (Li et al., 2018) and the feline MRV cross-reacts with three MRV serotypes based on the neutralization testing (Csiza, 1974). These facts suggest a potential serological cross-reaction among different serotypes of MRVs. Therefore, whether the high sero-prevalence in pigs we found is MRV1-specific or due to cross-reactivity with other serotypes, or due to maternal antibodies needs to be determined in future studies.

In conclusion, we isolate and characterize a novel reassortant MRV virus that is pathogenic and transmissible in pigs although we did not reproduce the neurological disease in pigs. Our results combined with previous studies indicate that MRV is an important pathogen for the swine industry (Thimmasandra Narayanappa et al., 2015). Therefore, further surveillance and pathogenicity studies on MRVs in pigs should be performed in order to understand viral pathogenicity and transmissibility as well as reassortment of MRVs as the novel reassortant MRV might emerge to threaten animal and public health.

1. A composition comprising:
at least two segments or portions thereof of orothoreovirus, wherein said segment is selected from the group consisting of L1, L2, L3, M1, M2, M3, S1, S2, S3, S4, of orthoreovirus and any combination thereof and wherein said portion thereof is selected from the group consisting of λ1, λ2, λ3, μ1, μ2, σ1, σ2, σ3, μNS, μNSC, σNS, σ1s of orthoreovirus; and
an additional component selected from the group consisting of a stabilizer, an adjuvant, an antimicrobial, an antifungal, a preservative, and any combination thereof;
wherein at least one segment or portion thereof is from a different serotype, strain, or host than another segment or portion thereof.

2. The composition of claim 1, wherein the composition comprises 10 segments.

3. The composition of claim 2, wherein the composition comprises 3 large segments, 3 medium segments, and 4 small segments.

4. The composition of claim 3, wherein said large segments comprise the L1, L2, and L3 segments, or wherein said medium segments comprise the M1, M2, and M3 segments, or wherein said small segments comprise the S1, S2, S3, and S4 segments.

5. (canceled)

6. (canceled)

7. The composition of claim 1, wherein said portion thereof includes regions encoding for 8 structural proteins.

8. (canceled)

9. The composition of claim 1, wherein said portion thereof includes regions encoding for 4 nonstructural proteins.

10. (canceled)

11. The composition of claim 1, wherein said segment has at least 80% sequence identity with a sequence selected from the group consisting of SEQ ID NOs. 4-13 or includes a portion thereof that encodes an amino acid sequence having at least 80% sequence identity with a sequence selected from the group consisting of SEQ ID NOs. 14-25.

12. (canceled)

13. (canceled)

14. (canceled)

15. The composition of claim 1, wherein said composition comprises the protein encoded by the segment(s) or portion(s) thereof.

16. The composition of claim 1, wherein said segments or portions of thereof of orothoreovirus are in
a killed or inactivated orthoreovirus;
and wherein at least one segment or portion thereof is from a different serotype, strain, or host than another segment or portion thereof.

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. A method of reducing the incidence and/or severity of at least one clinical or subclinical sign of infection with orthoreovirus comprising the step of administering the composition of claim 1 to a subject or group of subjects in need thereof.

32. The method of claim 31, wherein the orthoreovirus comprises 10 segments.

33. The method of claim 31, wherein the orthoreovirus comprises 3 large segments, 3 medium segments, and 4 small segments.

34. The method of claim 33, wherein said large segments comprise the L1, L2, and L3 segments, or wherein said medium segments comprise the M1, M2, and M3 segments, or wherein said small segments comprise the S1, S2, S3, and S4 segments.

35. (canceled)

36. (canceled)

37. The method of claim 31, wherein said portion thereof includes regions encoding for 8 structural proteins.

38. (canceled)

39. The method of claim 31, wherein said portion thereof includes regions encoding for 4 nonstructural proteins.

40. (canceled)

41. The method of claim 31, wherein said segment has at least 80% sequence identity with a sequence selected from the group consisting of SEQ ID NOs. 4-13 or includes a portion thereof that encodes an amino acid sequence having at least 80% sequence identity with a sequence selected from the group consisting of SEQ ID NOs. 14-25.

42. (canceled)

43. (canceled)

44. (canceled)

45. The method of claim 31, wherein said composition comprises the protein encoded by the segment(s) or portion(s) thereof.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. A method of treating or preventing orthoreovirus comprising the step of administering the composition of claim 1 to a subject or group of subjects in need thereof.

62. The method of claim 61, wherein the orthoreovirus comprises 10 segments.

63. The method of claim 61, wherein the orthoreovirus comprises 3 large segments, 3 medium segments, and 4 small segments.

64. The method of claim 63, wherein said large segments comprise the L1, L2, and L3 segments, or wherein said medium segments comprise the M1, M2, and M3 segments, or wherein said small segments comprise the S1, S2, S3, and S4 segments.

65. (canceled)

66. (canceled)

67. The method of claim 61, wherein said portion thereof includes regions encoding for 8 structural proteins.

68. (canceled)

69. The method of claim 61, wherein said portion thereof includes regions encoding for 4 nonstructural proteins.

70. (canceled)

71. The method of claim 61, wherein said segment has at least 80% sequence identity with a sequence selected from the group consisting of SEQ ID NOs. 4-13 or includes a portion thereof that encodes an amino acid sequence having at least 80% sequence identity with a sequence selected from the group consisting of SEQ ID NOs. 14-25.

72. (canceled)

73. (canceled)

74. (canceled)

75. The method of claim 61, wherein said composition comprises the protein encoded by the segment(s) or portion(s) thereof.


SEQUENCE LISTING (25 sequences)

SEQ ID NO: 1 - DNA, 23 bp, Artificial Sequence
  probes/primers
tggcagcgdtggatacgttattc

SEQ ID NO: 2 - DNA, 22 bp, Artificial Sequence
  probes/primers
gcgaaytcttcagcrgaggagc

SEQ ID NO: 3 - DNA, 24 bp, Artificial Sequence
  probes/primers
cgtgaraaagcacagcataragcc

SEQ ID NO: 4 - DNA, 3846 bp, Mammalian Orthoreovirus
gctacacgttccacgacaatgtcgtccatgatactgactcagtttagaccgttcattgaaagcatctcaggtattactgaccaatcgaatgacgtgtttgaagatgcagcaaaagcattctcttcgtttactcgcagcgatgtctacaaagcgctggatgagatacctttctctgatgatgcgatgcttccaatccctccaactatatatactaaaccatctcacgattcatactattatattgatgctctaaaccgcgtacgtcgtagaacatatcagggccctgatgacgtgtacgtacctaattgttctatcgttgagttgctggagccgcatgagactctgacatcttatgggcgtttgtctgaagcgattgaaaatcgtgccaaggatggagacagccacgccagaattgcgacaacatacggtagaatcgctgaatctcaagctcgacagattaaggctccattggagaagtttgtgttggcactattagtgtctgaagcgggggggtccttatatgatccagttctgcagaagtatgatgagattccagatctgtcgcataattgccctttatggtgttttagggaaatctgtcgtcacatatctggtccattaccagaccgggcaccttatctttatttatcagcaggggttttctggttaatgtcaccacggatgacgtctgcgatccctccgttactatctgatcttgttaacttagctattttgcagcagactgcgggtttggatccatcattagtgaaactgggagtgcagatatgccttcacgcagcagctagctcaagttatgcgtggtttattctaaagactaagtccatttttcctcaaaacacgctacatagtatgtatgaatctctagaaggggggtattgtcctaaccttgaatggttagagcctagatcggactataagtttatgtacatgggagtcatgccattgtccactaagtatgctaggtcggcaccgtctaatgaaaagaaggcgcgagaacttggcgaaaaatatggattgagttcagtcgtcagtgagcttcgtaaacggacagcggcctatgttaaacatgattttgcttcggtaaggtacattcgtgacgctatggcatgtactagcggcatttttcttgtaaggacacccactgaaacggtattgcaagaatatacacaaagtcctgagattaaagttcctatcccccaaaaagactggacaggccctgtaggtgaaattagaattctaaaagacacaactagctccatcgcgcgctacttgtatagaacatggtacttagcagcggcaagaatggcggctcagccacgcacatgggatccattgttccaggcgattatgagatctcaatacgtgacagctaggggtgggtctggcgcagcactccgcgagtctttgtatgcgatcaatgtgtcgttacctgattttaagggcctaccagtgaaggcagcaactaagatatttcaggcggcacaattagcgaacctgccgttctcccacacatcagtggctatactagctgacacttcgatgggattgcgaaaccaggtgcagaggcggccacgatccatcatgcccttaaatgtgccccaacagcaggtttcagcgccccatacactgaccgctgattacatcaattatcacatgaatctgtcgactacgtctggtagtgcggtcattgagaaggtgattcctttaggtgtatacgcttcaagccctcctaaccaatcgattaacattgacatatctgcgtgtgacgcgagtataacttgggacttctttctatctgtgattatggcggctatacacgaaggtgttgccagtagttccattggaaaaccgtttatgggagttcctgcatccattgtaaatgatgagtctgtcgttggagtgagagctgctaggccgatatcaggaatgcaaaacatggttcaacatctatcaaaactttataaacgcggattttcatacagagtgaacgattctttttctccaggcaacgattttactcatatgactaccacttttccgtcaggttcgacagccacctctactgagcacactgccaataatagtacgatgatggagactttcctgacagtatggggaccagaacatactgacgaccccgacgtcttacgtctaatgaagtctttaactattcaaaggaattacgtgtgtcagggtgatgatggattgatgattatcgatgggaatactgctggtaaggtgaaaagtgagactattcagaagatgctggagttaatctcaagatatggtgaggagtttggatggaaatatgacatagcgtacgatgggactgccgaatacctaaagctatatttcatatttggctgtcgaattccaaatcttagtcgtcatccaatcgttggaaaagaacgggcgaattcttcagcagaggagccctggccagcaattttagaccagattatgggtatcttctttaatggcgttcatgacgggttacagtggcagcggtggatacgttattcatgggctctatgctgtgctttctcacgtcaaagaacaatgattggtgagagcgtgggctacattcaatatcctatgtggtcatttgtctactggggattaccactggtaaaagtgtttgggtcagacccatggatattttcttggtacatgccgactggagacctgggaatgtatagttggattagtctaatacgacctctaatgacaagatggatggtagctaatggttacgtgactgacagatgctcacccgtattcgggaatgcagattatcgcaaatgtttcaatgagattaaactatatcaaggttattatatggcacaattgcctaggaatcccacgaaatctggacgagcggctcctcgggaggtgagagagcaattcactcaggcactatccgattatttgatgcaaaatccagaactgaagtcacgtgtgctgcgtggtcgtagtgagtgggagaagtatggagcggggataattcacaatcctccatcgctattcgatgtaccccataagtggtatcagggtgcacaagaggcggcaaccgctacgagagaagagctggcagaaatggatgagatattaatgcgcgctcgaaggcacagttattcgggtttttcaaagttgttggaggcatacctgctcgtgaaatggcgaatgtgcgaggcccgtgaaccggcggttgatttgcgattaccattgtgtgcgggtattgacccactaaactcagatccttttctcaagatggtaagcgttggaccaatgcttcagagtacgcgaaagtactttgctcagacactattcatggcgaagacggtgtcgggtcttgacgttaacgcgattgacagcgcgttgttacgactgcgaacattgggtgctgacaagaaggcattaacagcgcagttattaatggtgggacttcaggagtcagaggcggacgcactggctgggaagataatgttgcaagatgtgagtactgtgcaattagccagagtggtcaatttagctgtgccagacacctggatgtcgttggattttgactccatgttcaaacaccatgtcaaattacttcccaaagatggacgccatctaaacactgacattccccctcgcatgggatggttacgagctattttacgattcttaggtgctggaatggtaatgactgcaactggagttgctgttgacatatatctggaggatatacacggtggtggtcgatcacttggacagagattcatgacttggatgcggcaggaaggacggtcggcgtgagtctaccatgggtcgtggtgcgtc

SEQ ID NO: 5 - DNA, 3902 bp, Mammalian Orthoreovirus
gcaatggcgaacgtttggggagtgagacttgcagactcgttatcatcacccactattgagacaagaactcgtccttacacactacgcgatttctgttccgacttggatgctgtggctggcaaggaaccctggagaccattacgtaatcagaggacgaatgacattgtcgccgttcaattgtttaggccactgcaaggactggtgcttgacactcagttctatggattccctggcgttttttcagaatgggagcagtttatgaaggcgaaactccgtgtgttgaaatatgaggttttgcggatctacccgatcagtaattataatcatgagtgtgtcaacgtttttgtagcgaatgctcttgtcggggcgttcctgtccaatcaagccttctatgacttgttgcctctattagtaataaatgatactatgataaatgacttacttgggacaggtgccgccctttctcaatttttccaatctcatggtgaggtcttagaagttgccgcaggaaggaagtacctgcaaatgaagaactactcgaacgatgacgatgatccacctttgttcgccaaagatctgtcggattatgcgaaggcgttttacagtgacacatttgagactctagaccgatttttctggacacatgactcgtctgcgggcgttctagtgcattatgataagcccactaacgggaatcattacatcttgggtactttaacacagatggttagtgctcctccgcatattattaacgctactgatgcgttattgctcgaatcgtgcttagagcaatttgcggcgaatgtgagagccaggccagcgcagcctgttacgagattggatcaatgttatcatttacggtggggtgctcaatatgttggtgaggactcattgacgtaccgtttgggggtgctctcgctgctggctaccaacggatatcaactagctagaccgatccctaagcaattgacgaatcggtggctctctagttttgtcagtcaggtaatggcggatggtgtaaatgagacgccattatggcctcaagagagatatgttcaaatagcttacgattcaccatctgtagtcgatggagctacgcactatggctatgtcagactaaatcagttacggttgggcatgagggtgtccgctcttcagtcgttgagtgatactccagctccgatacagtggttgccgcagtatactattgaacaggcagctattgacgaaggcgatctaatggtttctcgcttgactcaactaccattacgtcctgactatggtagcatatgggttggtgatgctctatcgtattatgttgattacaatcgtagccatagagtcgtgctatcatccgagttgccacagctaccagatacatactttgacggagacgagcaatacggtcgtagcttgttctctctagctcgaaaaattggtgatcgatctctcatcaaagatacggcagtgcttaagcatgcgtatcaggccatcgatccaaacactggaagggaataccttcgcgcagggcagtccgttgcatacttcggggcatcagctggtcattcgggagcggatcaacctctggtaattgagccatggacgcagggtaagattagtggtgtgcctccgccctcttcagttaggcagttcgggtatgatgttgctaaaggtgcgatcgtagacttagcaagaccgtttccgtcgggcgactaccaatttgtatattctgacgtcgatcaggtcgttgatggccacgatgatctcagcatatcctcaggactggtggagagtctattagactcatgcatgcatgccacatccccaggtgggtcattcgtgatgaagattaacttcccgacacgtactgtctggcactatatagrgcagaagattctcccaaatattacctcgtacatgttgattaaaccatttgtgactaacaatgtagaattattctttgtggctttcggtgtgcatcaacaatcagcgttaacatggacgtctggggtgtatttcttcctggtcgatcatttctatcgatacgagacactgtctacgatttcacgtcagttgccatcattcggatacgttgatgatgggtcgtccgtgacaggcattgaaatgatcagtcttgaaaatccaggcttttcaaatatgacccaagctgcacgtgttggaatatccgggttgtgtgcgaatgttggtaatgcgcgtaaattaatatctatccatgaatctcacggggcacgcgtgctcaccatcacatcaagaagatctccagcttcggccaggcggaaggctcgcttacgctatttgccacttgtagacccacgatctttggaagtgcaggcccgtacgatactaccatctaacccagtattgtttgataacgtgaacggagcatcgcctcatgtatgtttgacgatgatgtataactttgaggtgtctagtgcggtatatgatggtgatgtggtacttgaccttggtaccggtcctgaagcgaagattctggagctgattcctccaacatccccggtaacatgcgtagacattaggccaactgcacagccaagtggctgttggaacgtacgtacgacttttctggagcttgattacttaagtgacggttggataacgggtgtacgtggcgacatcgtcacttgtatgctgtctctgggtgctgctgctgctggaaaatccatgacgttcgacgcggcatttcaacagctagtgaaagttcttactaaaagcacagctaacgtgttgttgattcaagtcaattgcccaacagatgtgatccgaacaattaagggatatttggagatcgatcaaactaataagcgataccgatttcccaagtttggccgcgatgaaccgtattctgacatggattccttagagcgcatatgtcggactgcatggccaaattgttctatcacgtgggtgcccttgccctatgacttgcgttggactaagcttgctttacttgagtcgaccacattgagcagtgcgtcggtgagaattgctgaattgatgtacaagtacatgcccgtgatgagaatagatattcatgggttacctatggaaaagcaaggcagtttcatagtgggtcagaactgctctttgactataccgggcttcaacgcacaggacgtattcaattgttatttcaactccgcgcttgccttctctactgaggatgttagttctgcaatgataccgcaggtggcggctcagtttaatgctaacaaaggagagtggtcattggacatggtgttctcagacgctggtatctacacgatgcaggcattggtcggttccaacgcaagtcctgtgtctctgggttcgtttgtagtggactctccggatgtggacataacagatgcatggcctgctcagctggacttcaccatagctggcactgacgtcaacattacggttaatccttattaccgcttgatggcctttgtgaaaattgatggacaatggcagattgcgaatcctgataagttccaattcttctcatcaagtacagggacgttggtgatgaatgtgaagttagatatagctgataggtacttattatactacattcgtgacgtgcaatctagagaggtggggttttacatacagcacccgttgcggttattaaataccattacgctgcctacaaatgaggacttattcttgagcgctccggacatgcgcgagtgggcagtaaaggagagtggtaataccatatgcatactcaacagtcaaggttttgtgccgcctcaggattgggatattcttaccgataccattagctggtctccttcgcttccaacttacgtggtacctccgggtgattataccctgacacctctgtaactcattacccctcgtgagcgtgcctaatt

SEQ ID NO: 6 - DNA, 3864 bp, Mammalian Orthoreovirus
gctaatcgtcaggatgaagcggattccaaggaagacaaagggcaaatccagcggaaagggcaatgactcaactgatagagcggacgatggctcgagccaattacgagacaagcaaaacaataagaccggccccgccactacagagcctggaatgtccaaccgagagcaatacaaagctcgaccaagtattgcatctgtgcagagggccactgagagtgcagagctgcccattaagaataacgacgagggaacgccagataagaaaggaaacactaagggagactcaattagtgagcacagtgaggctaaagacgaagcggatgaagcgacgaagaagcaggcaaaagatacagataaaggtaaggcgcaagtcacatattcagacactggtattaacaatgctaatgaactgtcaaggtctgggaatgtggataatgagggtggaagtaatcagaagccgatgtccaccaggatagctgaagcaacgtctgctatagtgtcgaaacatcctgcgcgtgttgggttaccacctaccgccagcagtggtcatggatatcagtgtcatgtctgttctgcagtcctgtttagtcctttagacctagacgctcacgtcgcctcacatggtttgcatggtaatatgacattgacatcgagtgagattcagcgacatatcactgagtttatcagttcatggcaaaatcatcctattgttcaagtttcggctgacgtcgaaaataagaagactgctcaattgctgcacgctgacactcctcgacttgtcacttgggatgccggtttgtgtacttcgttcaaaatcgttccgattgtaccagctcaggtgccgcaggatgtgctagcctatacgttctttacctcatcatatgctattcaatcaccgtttccagaggcggcagtgtctaggattgtggtgcatacaagatgggcatctaatgttgactttgaccgagattcgtctgtcgtcatggcaccacccacagaaaacaatatccatttgtttaaacagttgctgaatactgacaccctgtctgtgaggggggctaacccgctaatgtttagagcgaacgtattacatatgttgctggagttcgtattggataacttgtacctgaacagacatacgggattctctcaagaccacacgccatttactgagggcgctaatctgcgttcacttcctggccccgacgctgagaaatggtattcgattatgtatccaacgcgtatgggaacaccgaatgtatcgaaaatatgtaacttcgtcgcctcttgtgtgcgaaatcgagtcgggaggtttgatcgggcgcagatgatgaacggagccatgtcagagtgggtggatgtcttcgagacttcagacgcacttaccgtctccattcgaggccgatggatggctagactagctcgcatgaacataaatccaacagagatcgaatgggcgttgactgaatgtgcacagggatatgtgactgtcacaagtccttacgctcctagtgtaaatagattgatgccatatcgtgtctccaacactgagcggcagatatcacagataatcaggatcatgaacattggcaataatgcgacggtgatacaacccgttctgcaagatatttcggtacttcttcaacgcatatcaccacttcagatagatccaaccattatttccaacactatgtcaacagtctcggagtctactactcagacactcagccccgcgtcctcaattttgggtaaactacgaccgagtaactcagatttctctagttttagagttgcgttggctggatggctttataatggagttgtgacgacggtgattgatgatagttcatatccaaaggacggtggcagcgtgacctcacttgaaaatctgtgggatttcttcatccttgcgcttgccctaccactgacaactgacccatgtgcacctgtgaaggcgttcatgactttagccaatatgatggtcggcttcgagacaatccctatggataatcagatctacactcaatcgagacgcgcgagtgctttctcaacgcctcacacgtggccacgatgcttcatgaacatccagttaatttctcccatcgatgctccaatcttacgacagtgggctgaaatcattcatagatactggcctaacccttcacagattcgttatggtgcgccgaacgtttttggctcggcaaatctgttcactccacctgaggtgctgttattgccaatcgatcatcaaccagctaatgtgacgacgccaacgttggacttcaccaacgagttgaccaattggcgcgctcgtgtttgtgagcttatgaagaatcttgttgataatcaaagatatcaacctggatggacgcaaagtctagtctcgtcaatgcgtggaacactggacaaattgaaattgatcaaatcgatgacaccaatgtatctgcaacagctagctccggtagagttagcagtgatagctcccatgttgcctttcccacctttccaggtgccctacgtccgcctcgatcgtgatagaattccaacaatggtcggagtgacacgacagtcacgggacactatctctcagccagcgctatcactgtcgacaaccaataccactgtcggtgtgcctctagctctagacgcaagggctattaccgtcgcgctgttgtcagggaaatatccgccggatctagtgacaaatgtatggtacgctgatgccatttatccaatgtatgcggatactgaggtgttctccaatcttcagagggatatgattacctgcgaagccgtgcagacactagtgactctagtggcgcagatatcagagacccagtatcctgtggataggtatcttgattggatcccatcactgagagcatcggcggcgacggcggcgacatttgctgagtgggttaatacttcaatgaagacagcgtttgatttgtctgacatgctattagagcctctcctaagcggggacccgaggatgactcaactagcgattcagtatcagcaatacaatggcagaacgtttaatgtcgtacctgaaatgccaggttcagtcattgctgactgcgttcaactgacagcagaagtcttcaatcacgaatataacctgtttgggattgcgaggggtgatatcatcattggccgtgtccagtcgacacatttgtggtcaccgctggctcctccacctgacctggtgtttgatcgtgatactcctggcgttcacatcttcggacgagattgccgtatatcgtttggaatgaatggcgccgcgccaatgattagagatgagactggcatgatggtgcctttcgaaggaaattggattttcccactggcgctttggcaaatgaatacacgatattttaatcaacagtttgacgcgtggattaagacaggagagttgcgaatccgtattgagatgggcgcgtatccatatatgctgcattactatgatccgcgtcagtatgctaatgcatggaatctgacatcagcctggcttgaagaaattacaccgacgagcattccatccgtgcctttcatggtgccaatttcaagtgatcatgacatttcctctgctccagctgtccaatatatcatttcgactgaatacaatgaccggtccttattctgcactaattcatcatctccccaaactatcgctggaccagacaaacacattccagttgagagatataacattctgaccaaccctgatgctccacccacgcagatacaactgcccgaagttattgatttgtacaacgtcgttacacgctatgcgtatgagactccacctattaccgctgttgttatgggtgttccttgatcctcatcctcccaacgggtgct

SEQ ID NO: 7 - DNA, 2301 bp, Mammalian Orthoreovirus
gctattcgcggtcatggcttacatcgcagttcctgcggtggtgggttcacgtgcaagtgaggctattggattgctagaatcgtttggagtagacgctggggctgatacgaatgatgtttcatatcaagatcatgactatgttttggatcagttacagtatatgttagatggatatgaggctggtgacgtcatcgatgcactcgtccacaagaattggttgcactattctgtctattgcttgttacctcccaaaagtcaactactagagtattggaaaagcaatccttcagcgataccggacaacgttgatcgtcgacttcgtaaacggttaatgctaaagaaagatctcagaaaagatgatgaatacaatcaattggcgcgtgctttcaagatatcggatgtctacgcacccctcatttcatctacgacgtcaccgatgacaatgatccagaacttgaatcagggcgagatcgtgtacaccacgacggacagggtaattggggctagaatcttgttatatgctcctagaaagtactatgcgtcaactctatcatttactatgactaagtgcatcattccgtttggcaaagaggtgggccgggctcctcactctagatttaatgttggcacattcccatcaattgccaccccgaagtgttttgtcatgagtgggattgatgttgagtccataccaaatgaattcatcaagttgttttaccaacgtgtcaagagtgttcacgctaatatactaaatgacatatcacctcagatcctttctgacatgataaacaggaagcgtttgcgcgttcatactccgtcagatcgccgagccgcgcagttgatgcatttgccctaccatgttaagcgaggggcgtctcatgtcgacgtttataaggtggatgttgtggacgtattgtttgaggtagtggatgtggccgatgggttgcgcaacgtatctaggaaactaactatgcacactgttccggtatgtattctcgaaatgttgggtattgagattgcagactattgcgttcgtcgagaggatggaatgttcatagattggtttttgcttttgaccatgctatctgatggcttaactgatagaaggacgcgttgtcaatacttgattaatccgtcaagcgtgcctcctgatgtgatacttaacatctctattactggatttatcaataggcatacgattgacgtcatgcctgacacatacgacttcgttaaacccattggcgccgtgctgcctaagggatcatttaaatcaacaattatgagagttcttgactcaatatcaatattaggagtccagatcatgccacgcgcgcatgtagtcgattcggatgaggtgggtgagcaaatggagcctacgtttgagcatgcggtcatggagatatacaaagggattgctggcgttgattcgctggatgacctcatcaagtgggtgctgaactcggatctcattccgcatgatgacaggcttggccacttatttcaagcgtttctgcctctcgcaaaagacttgttagctccaatggctagaaagttttatgataactcaatgagtgagggtagattgctgacattcgctcatgctgacagtgagttactgaacgcaaactactttggtcatttattgcgactaaaaataccatatattacggaggttaacctaatgattcgcaagaatcgtgaaggtggagagctatttcagcttgtgttatcacatctatataaaatgtatgctactagcgcgcagcctaaatggtttggatcattattgcgactgttaatatgcccctggttgcacatggagaaattgataggagaagcagacccagcatctacgtcggctgagattggatggtatatccctcgtgaacagctgatgcaagatggatggtgcggatgtgaagatggattcattccctatgttcgcatacgtgcgccaaagctggttatagaggagttagcggaaaagaactggggccaatatcatgcgcaagttattatcactgatcagctcgtcgtaggcgaaccgcgtagggtatctgccaaggctgtgatcaaaggcaaccacttaccagttaagttagtctcacgatttgcatgtttcacattgacgacgaagtatgagatgaggctttcatgcggccatagcactggacggggggctgcatacaatgcgagactagctttccgatctgacttggcgtgatccgtgacatgcgtagtgtgacacctgcccctaggtcaatgggggtaaggggcgggctaggactacgtacgcgcttc

SEQ ID NO: 8 - DNA, 2203 bp, Mammalian Orthoreovirus
gctaatctgctgaccgttactctgcaaacatggggaacgcttcatccattgttcagacaatcaatgtcacaggagatggcaatgtcttcaagccgtcagctgaaacgtcttcaacggctgtaccctcattgagtctttcacctgggatgctcaatcctggcggtgtaccatggatcgccatcggggacgaaacgtcggtgacttcgccgggtgccttgcgtcggatgacatctagagacataccagaaacagctattatcaacactgataattcatctggggctgtgccgagcgagtccgctttagtgccatacaatgatgagccgctggtagttgtcactgagcatgcaatcgctaactttactaaagctgagatggccttagaattcaacagagagtttttagacaaactccgggtgttatccgtatcaccgaagtattccgatctgttgacgtatgtcgattgttacgttggagtctccgcgcgtcaggcactcaataacttccagaaacaggtgcctgtgataaccccaacacgtcagacaatgtatgtggattccatccaagcggcattgaaagcgttagagaaatgggaaattgatttacgagtcgcgcagacattactaccaacaaacgtgccgattggtgaggtctcatgtcccatgcagtcagtggttaagttgctagatgatcagctaccagatgacagtttgatcagaaggtatccgcgagaggcggcggtagcactggctaaaagaaatggagggatacaatggatggatgtttcagagggcactgtcatgaacgaggctgttaacgcggtagccgctagcgcactagctccttctgcatcctcgccaccacttgaagaaaagtcaaagctgacagaacaggccatggatttagttacagccgcagagccagaaataattgcatccttggttcctgtaccggctccggtctttgccatccctccaaagccagctgattataacgtgagaactttgaaaattgatgaggcaacgtggctgagaatgattccgaagacgatgaccactccattccaaattcaagtcacggataatactagcaccaggtggcacatgaatttaaggggtggaacacgggttgtcaatctagaccagattgccccaatgcggtttgtgcttgatttggggggtaagagttataaagagacgagttggaatcctaacgggaagagagttggcttcattgttttccagtccaaaatacctttcgagctgtggacggctgcatcgcaaatagggcaagcaacagttgttaactacgttcagctgtatgccgaagatagttcatttacagatcagtccatcattgctactacttcattggcgtataattacgaaccagagcagttgaacaaaacggatcctgaaatgaattactacttgcttgccacattcattgattcagcagaaatcaatccttctaatatgactcagccggatgtgtgggatgctttgctaaccatgtcaccattgtctgctggtgaagtgactgtgaaaggggccgtagttagtgaggtaacaccagcggatctggtcggaagttacacaccggaatcacttaacgcctcattacctaatgatgccgcgcgctgcatgattgacagggcttcaaaaatcgcggaagctattaagatcgatgatgatgctggtccagatgagtattctcctaattctgtgccgatccaaggacagttggcgatatctcagcttgaaaccggctatggtgttaggatctttaacccgaaaggtattctgtccaagatagcgtcgcgcgctatgcaagcattcattggagacccaagtactatcattactcaggctgctcctgtgctttctgacaagaacaactggatagcgctggcgcaaggggtgaagacgagcttgcgtacaaagagcctttccgcgggagttaagacggccgttagcaagttgagttcatcagaatctattcaatcttggactcaagggtttctggacaaagtctctactcatttcccagcgccaaggtctgattgccctcagtctggagacagtgatggtgggtctgctagacggctgaagcgggactcatacgcaggagtggttaagcgtgggtatacgcgctagaccactcaccctggtgacgcgggggtaagggacgcaggaaaatcatc

SEQ ID NO: 9 - DNA, 2241 bp, Mammalian Orthoreovirus
gctaaagtgaccgtggtcatggcttcgttcaagggattttccgccaacactgttccagtttctaaggccaaacgtgacatatcagcccttgctgctactcctggatttcattcacaatccttcactccgtctgtggatatgtctcaatcgcgtgagttccttacaaaagcgatcgagcaagggtctatgtctataccttaccagcatgtgaatgtaccgaaagttgatcgtaaagttgttagcttggtggtgcgacctttttcttcaggtgctttctctatctctggagtcatttcgccagctcatgcctatctactagattgtctacctcagcttgaacaggcgatggcttttgttgcttcacccgaatcttttcaggcttcagatgttgcaaagcgttttgctataaagccaggtatgagcctccaggatgccatcactgcgtttattaatttcgtgtctgcgatgttgaaaatgacggtaactcgtcagaattttgatgttattgtggctgagatcgagaggcttgcttcaaccagcgtgtctgccaggactgaagaagcgaaagtcgctgatgaggagctgatgttattcgggctagatcacagagggccacagcagttggatatttctgatgctaaggggataatgaaggctgttgacattcagacaactcatgatgtccatttggcacccggcgttggtaatattgaccctgaaatttataacgaagggcggttcatgtttatgcagcacaagccacttgcagcggatcaatcgtattttaccttggagactgcggattatttcaagatttatccaacatatgatgagcatgatggtaggatggctgaccaaaagcagtcaggattgatactatgtactaaagatgaagtgttggctgagcaaactatatttaaactggacactcctgacgataaaactgttcatctgctagatcgtgatgacgatcacgttgttgccaggtttaccaaagtatttatagaggatgtggctcccgggcatcatactgctcagcgatcgggacaacgctctgtgcttgatgatttatatgcgaatacgcaggtgatttccattacctccgctgctctgaagtgggtggtcaaacacggcgtgtctgatggaattgtgaacagaaagaatgtcaaggtgtgtgttggttttgaccccttgtacactctgtctacgcataacggaatatctctgtgtgccctgttaatggatgagaagctttcggtgctgaacagtgcgtgtcgtatgacgttacgctctctcatgaagactagacgtgatgctgatgcgcacagagcttttcaacgagttctttctcaaggatacacatcgctaatgtgctattatcacccttcacggaagctggcatatggtgaggtgctttttccagaacgatccaatgacgtggtcgatgggatcaagctacagttggacgcatctagacattgtcatgaatgtcctgtgttgcagcagaaagtggttgaattggaaaaacagatcgtcatgcaaaagtcgattcagtcagatactgctccaatagcgctgcaaccactgttgtctcagttgcgtgagctgtccagtgaagttactaggttacagatggagttgagtagggctcaatccctgaatgctcagctagaggcggatgtcaaatcagctcaatcatgcagcctggatatgtacctgagataccacacttgcattaatggtcatgctaaagaggatgaattgcttgatgctgtgcgtgtcgcgccggatgtgaggaggcagattatggacaggaggagtgaggtgagaaaggggtggtgcgaacgtatttctaaggaagcgtctgccgaatgtcaaactgttattgatgaactgactctgatgaatggaaagcaggcccaagagataatagagttacgtgattcggctgagaattatgagaaacagattgcggagctggtgagtaccatcacccaaaaccagataacttatcagcaagagttgcaagccttagtggcgaagaacgtggaattggacacattgaatcaacgtcaggctaggtcgttgcggattactccatctcttctatcagttactcctaccgactcagttgatggcgccgctgacctaatcgatttctctgttccaactgatgagctgtaaatgatccgtgatgcagtgttgtcctaatcccttaagccttcccgaccccaactcatc

SEQ ID NO: 10 - DNA, 1456 bp, Mammalian Orthoreovirus
gctattcgcgcctatggatgcgtctctcatttcagagatacggagaattgtacttcaactgacgcagtcgaacaatgggtcgatcaacaaggaactcaaagaaatcaaagaacaagtcgagatcaacgctactgatatcaggaataccaatcttaagctcgatgggatcagaggacaagttactagcatcagtgatagcatcgcagccattgaacgtagattgggtgaagtggatggtagatttgtggttatcgcgaatcagattacgcaactatctaactcagttggccagaacactcaagacatatccgccttgggtgacagactcaatgttgttgaaccaagaattgatgacttggatacattcgcatctaatctcgttggacgaacatccactttggaggcagatgttagaggaatccgtactgaactgtccgcattaacgacaagagtaactacggaagtagcgaggctagatgctctgattagcgccggtcaatcatccttgacgagtctatcatcaagattggatgcggttgaggctgcgatgattaccacggctggccgaggcttaaggaaggatggaaatacgttgaacataattgtaggtgatgggatgtggttcgataactccaatcagctgcagttagacttatcgaatcaagccaaaggagttggtttcgagggtggtggtgtggtggcgaagattgacacgaattatttttcatataacagtaatggtgaaatcacacttgtgggccagatcaatggtttacctgccagagtgtctacattggaatctttaaagattgacactgttttgcctcccttgaccgtgtctgaagcggatggtgccaggaatctgggcttgggttatgaaacagatgacttcactgttacaaattccatgttgtctctacgttctaggctaactttgccgacataccgttatcccttggagttagatgcttctattaaccgtgtccagatagctgaacgttttggaatgcgcgtagggagctggactggacaattacaatatcaacatccgcaattgaattggcaggcaaatgtgaccattaatctgatgaaggtggatgattggctggtgctcagtttcacgcaaatgacgacaagtggcattatggcggatgggaaatttacaattaatttttccgctggattatcttcggggtggcagacgggagagacacaaccttcgtcaataacagatccattatctacaacatttgctgctgtacaatatctcaaagccaatgggactcgtgtggatgcattcaggattatgggcgtatcagagtgggttgacggagagctagagataaagaattatgggggcacgtatacaggacatacgcaggtatattgggctccatggacgataatgtatccttgtaatgtgagataaatctagcgcgttccctcggcacaaggg

SEQ ID NO: 11 - DNA, 1330 bp, Mammalian Orthoreovirus
gctattcgctggtcagttatggctcgcgctgcgttcctattcaagactgttggattcggtggcctgcaaaatgtgccaattaatgatgagttgtcgtcacatctgcttcgagccggtaattcgccatggcagctgactcagttcttagattggataagtcttggaagaggattagctacatcagctcttgttccaaccgctggttcaagatactatcagatgagctgtttactgagtggcactctccaaattccgtttcgtcctaatcatcgatggggagatgctaggtttctgcgtctagtgtggtcagctcctacgcttgacgggttggttgttgccccaccacaggtcttagctcagccggcgttacaggcgcaggcggatcgagtgtatgattgtgatgattacccattcttggcacgtgacccgcgattcaaacatcgagtgtatcagcaattgagtgccgtgactctgctcaacctgacgggatttgggccaatttcctatgttcgagtagacgaagccatgtggagtggagatgtgaaccagcttctcatgaactacttcgggcacacatttgccgaaattgcatatacattatgtcaggcttcagccaacagaccttgggagcacgatggtacgtacgcgaggatgactcaaatcatactgtctttgttctggttatcgtatgtcggtgtaattcatcagcagaacacttaccggacgttctatttccaatgtaatcggcgcggtgacgctgctgaagtgtggattctttcctgctcactaaaccactccgcccaaattaggccgggtaatcgcagtctattcgtcatgccaacaagtccagactggaatatggacgtcaatctaattttaagttcgacgttgacagggtgcttgtgttcgagctctcagttaccgctcattgacaataactcagtgcctgcggtttcgcggaacatccacggctggactggtagagctggtaaccaactgcacggtttccaagtgcgacgaatggtgactgaattctgtgacagattaagacgcgatggggtcatgactcaagctcaacagaatcaagttgaagcgctggcagatcaaactcaacaattcaagagggatcaacttgaggcctgggctagggaagatgatcagtataatcaggctcatccgaattctccgatgttccgtacgaagccatttacgaatgcgcaatggggacgaggaaataccggagcgactagtgccgcaattgcagcccttatctaatcgtcttggagtgagggggtccccccacacccctcacgactgaccacacattcat

SEQ ID NO: 12 - DNA, 1195 bp, Mammalian Orthoreovirus
aaagtcacgcctgttgtcgtcactatggcttcctcactcagagcagcgatctctaagatcaagagagatgatgttggtcaacaagtttgtcccaattatgtcatgcttaggtcatcggtcacaacgaaagtggtacgaaacgttgttgagtatcaaatccgtacgggtggattcttttcgtgcctagctatgttaagaccgctccagtatgccaagcgtgagcgtctgcttggacaacggaatctggaacgcatatcaactagggacatcctccaaacacgcgacttacactctttatgcatgccaactcctgacgcaccaatgtccagtcatcaggcagtaaccatgagggagctgatttgcagctacttcaaggtcgaccacactgatgggttgaaatatatacccatggatgagaaatactctccatcgtcgctcgccaggctgtttactatgggtatggctggactacacattaccactgagccttcctacaaacgtgtgcccatcatgcacttggcagcagatttagactgcatgacgttagctttgccctacatgattacacttgatggtgacacggtggtacctgttgccccaacgctttctgcagaacagcttttggatgatggacttaaggggatggcgtgcatggacatctcatacggatgtgaggtggatgctaataaccgatcagctggtgatcagagcatggactcttcacgatgcatcaacgaattatattgcgaggagacggcagaagctatttgtgtactcaaaacatgtcttgtgctgaactgtatgcaattcaaacttgagatggatgatttagcacataacgctgctgagctggacaagatacagatgatgataccttttagtgaacgcgttttcagaatggcttctgcatttgctaccattgatgtccagtgtttcaggttttgtgtaatgatgaaggataagaatttgaagatagacatgcgcgaaacgatgagactttggactcgatcggcgctggacgagtcagtggttacgtcatctctgagtatttcgttggatcgaggcagatgggtggcggctgatactaatgatgctagattgctggtgtttccaattcgcgtgtaatgggtgagtgagccgatgtggtcgccaagacatgtgccggtgtcttggtggtgggtggcgcctaatcatc

SEQ ID NO: 13 - DNA, 1187 bp, Mammalian Orthoreovirus
gctatttttgcctcttcctagacgttgtcgcaatggaggtgtgcctacctaatggtcatcagatcgtcgactggatcaacaatgcattcgaaggacgggtatcgatttatagtgcacagcagggatgggataagacaatttcagctcagccggatatgatggtgtgtggtagcgctgtcgtttgcatgcactgtttaggtgtggttggatcattgcagcgaaagctgaaacatctgccccatcataaatgtaatcagcaattgcgtgagcaggattatgttgatctacaatttgctgatcgtgtaaccgctcactggaagcgtggcatgttatcatttgtatcccagatgcatgctgtcatgaacgatgtgacacctgaggagcttgaaagagtgaggaccgacggtgggatcttggctgagctcgactggcttaaagtggagtctggatcaatgtttcgttcgattcactcaagctggactgatccccttcaggtggtcgaagatctagatactcagctagatcgctattggacagccctgaatttgatgattgattcatcggatctagtgccgaacttcatgatgcgcgatccatcgcatgcttttaatggagtgaagctggagggtgaagcgcgacagactcaattcccgcgtacattcgattccgggtcgaatctgaagtggggtgttatggtttatgactattctgaacttgagagggattctcaaaaaggacgatcctataggaaagagatcgttacgccggcgaaagactttggccactttggcttgtcccattattcccgtgcgacgacaccgatccttggcaaaatgcctgccgtcttttctggtatgctgaccggaaactgtaagatgtatccgtttataaagggcactgctaagctgaaaacagtcaagaagttagttgatgctgtgaaccacacgtggggttttgagaagatcagatacgccttaggccctggtgggatgacgggatggtataataggactatgcagcaagcgccaattgtgctgactccagcggcactgactatgtttccggatatgaccagatttggcgatctacaatatccaatcacgattggcgatccggctgtccttgggtaaacatctccatcttctcagcgccaggcctgaccaacctggtgtgacgtgggacaggca

SEQ ID NO: 14 - protein, 1267 aa, Mammalian Orthoreovirus
MSSMILTQFRPFIESISGITDQSNDVFEDAAKAFSSFTRSDVYKALDEIPFSDDAMLPIPPTIYTKPSHDSYYYIDALNRVRRRTYQGPDDVYVPNCSIVELLEPHETLTSYGRLSEAIENRAKDGDSHARIATTYGRIAESQARQIKAPLEKFVLALLVSEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYAWFILKTKSIFPQNTLHSMYESLEGGYCPNLEWLEPRSDYKFMYMGVMPLSTKYARSAPSNEKKARELGEKYGLSSVVSELRKRTAAYVKHDFASVRYIRDAMACTSGIFLVRTPTETVLQEYTQSPEIKVPIPQKDWTGPVGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSTTSGSAVIEKVIPLGVYASSPPNQSINIDISACDASITWDFFLSVIMAAIHEGVASSSIGKPFMGVPASIVNDESVVGVRAARPISGMQNMVQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGNTAGKVKSETIQKMLELISRYGEEFGWKYDIAYDGTAEYLKLYFIFGCRIPNLSRHPIVGKERANSSAEEPWPAILDQIMGIFFNGVHDGLQWQRWIRYSWALCCAFSRQRTMIGESVGYIQYPMWSFVYWGLPLVKVFGSDPWIFSWYMPTGDLGMYSWISLIRPLMTRWMVANGYVTDRCSPVFGNADYRKCFNEIKLYQGYYMAQLPRNPTKSGRAAPREVREQFTQALSDYLMQNPELKSRVLRGRSEWEKYGAGIIHNPPSLFDVPHKWYQGAQEAATATREELAEMDEILMRARRHSYSGFSKLLEAYLLVKWRMCEAREPAVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVSTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 15 - protein, 1289 aa, Mammalian Orthoreovirus
  misc_feature (628)..(628): Xaa can be any naturally occurring amino acid
MANVWGVRLADSLSSPTIETRTRPYTLRDFCSDLDAVAGKEPWRPLRNQRTNDIVAVQLFRPLQGLVLDTQFYGFPGVFSEWEQFMKAKLRVLKYEVLRIYPISNYNHECVNVFVANALVGAFLSNQAFYDLLPLLVINDTMINDLLGTGAALSQFFQSHGEVLEVAAGRKYLQMKNYSNDDDDPPLFAKDLSDYAKAFYSDTFETLDRFFWTHDSSAGVLVHYDKPTNGNHYILGTLTQMVSAPPHIINATDALLLESCLEQFAANVRARPAQPVTRLDQCYHLRWGAQYVGEDSLTYRLGVLSLLATNGYQLARPIPKQLTNRWLSSFVSQVMADGVNETPLWPQERYVQIAYDSPSVVDGATHYGYVRLNQLRLGMRVSALQSLSDTPAPIQWLPQYTIEQAAIDEGDLMVSRLTQLPLRPDYGSIWVGDALSYYVDYNRSHRVVLSSELPQLPDTYFDGDEQYGRSLFSLARKIGDRSLIKDTAVLKHAYQAIDPNTGREYLRAGQSVAYFGASAGHSGADQPLVIEPWTQGKISGVPPPSSVRQFGYDVAKGAIVDLARPFPSGDYQFVYSDVDQVVDGHDDLSISSGLVESLLDSCMHATSPGGSFVMKINFPTRTVWHYIXQKILPNITSYMLIKPFVTNNVELFFVAFGVHQQSALTWTSGVYFFLVDHFYRYETLSTISRQLPSFGYVDDGSSVTGIEMISLENPGFSNMTQAARVGISGLCANVGNARKLISIHESHGARVLTITSRRSPASARRKARLRYLPLVDPRSLEVQARTILPSNPVLFDNVNGASPHVCLTMMYNFEVSSAVYDGDVVLDLGTGPEAKILELIPPTSPVTCVDIRPTAQPSGCWNVRTTFLELDYLSDGWITGVRGDIVTCMLSLGAAAAGKSMTFDAAFQQLVKVLTKSTANVLLIQVNCPTDVIRTIKGYLEIDQTNKRYRFPKFGRDEPYSDMDSLERICRTAWPNCSITWVPLPYDLRWTKLALLESTTLSSASVRIAELMYKYMPVMRIDIHGLPMEKQGSFIVGQNCSLTIPGFNAQDVFNCYFNSALAFSTEDVSSAMIPQVAAQFNANKGEWSLDMVFSDAGIYTMQALVGSNASPVSLGSFVVDSPDVDITDAWPAQLDFTIAGTDVNITVNPYYRLMAFVKIDGQWQIANPDKFQFFSSSTGTLVMNVKLDIADRYLLYYIRDVQSREVGFYIQHPLRLLNTITLPTNEDLFLSAPDMREWAVKESGNTICILNSQGFVPPQDWDILTDTISWSPSLPTYVVPPGDYTLTPL

SEQ ID NO: 16 - protein, 1275 aa, Mammalian Orthoreovirus
MKRIPRKTKGKSSGKGNDSTDRADDGSSQLRDKQNNKTGPATTEPGMSNREQYKARPSIASVQRATESAELPIKNNDEGTPDKKGNTKGDSISEHSEAKDEADEATKKQAKDTDKGKAQVTYSDTGINNANELSRSGNVDNEGGSNQKPMSTRIAEATSAIVSKHPARVGLPPTASSGHGYQCHVCSAVLFSPLDLDAHVASHGLHGNMTLTSSEIQRHITEFISSWQNHPIVQVSADVENKKTAQLLHADTPRLVTWDAGLCTSFKIVPIVPAQVPQDVLAYTFFTSSYAIQSPFPEAAVSRIVVHTRWASNVDFDRDSSVVMAPPTENNIHLFKQLLNTDTLSVRGANPLMFRANVLHMLLEFVLDNLYLNRHTGFSQDHTPFTEGANLRSLPGPDAEKWYSIMYPTRMGTPNVSKICNFVASCVRNRVGRFDRAQMMNGAMSEWVDVFETSDALTVSIRGRWMARLARMNINPTEIEWALTECAQGYVTVTSPYAPSVNRLMPYRVSNTERQISQIIRIMNIGNNATVIQPVLQDISVLLQRISPLQIDPTIISNTMSTVSESTTQTLSPASSILGKLRPSNSDFSSFRVALAGWLYNGVVTTVIDDSSYPKDGGSVTSLENLWDFFILALALPLTTDPCAPVKAFMTLANMMVGFETIPMDNQIYTQSRRASAFSTPHTWPRCFMNIQLISPIDAPILRQWAEIIHRYWPNPSQIRYGAPNVFGSANLFTPPEVLLLPIDHQPANVTTPTLDFTNELTNWRARVCELMKNLVDNQRYQPGWTQSLVSSMRGTLDKLKLIKSMTPMYLQQLAPVELAVIAPMLPFPPFQVPYVRLDRDRIPTMVGVTRQSRDTISQPALSLSTTNTTVGVPLALDARAITVALLSGKYPPDLVTNVWYADAIYPMYADTEVFSNLQRDMITCEAVQTLVTLVAQISETQYPVDRYLDWIPSLRASAATAATFAEWVNTSMKTAFDLSDMLLEPLLSGDPRMTQLAIQYQQYNGRTFNVVPEMPGSVIADCVQLTAEVFNHEYNLFGIARGDIIIGRVQSTHLWSPLAPPPDLVFDRDTPGVHIFGRDCRISFGMNGAAPMIRDETGMMVPFEGNWIFPLALWQMNTRYFNQQFDAWIKTGELRIRIEMGAYPYMLHYYDPRQYANAWNLTSAWLEEITPTSIPSVPFMVPISSDHDISSAPAVQYIISTEYNDRSLFCTNSSSPQTIAGPDKHIPVERYNILTNPDAPPTQIQLPEVIDLYNVVTRYAYETPPITAVVMGVP

SEQ ID NO: 17 - protein, 736 aa, Mammalian Orthoreovirus
MAYIAVPAVVGSRASEAIGLLESFGVDAGADTNDVSYQDHDYVLDQLQYMLDGYEAGDVIDALVHKNWLHYSVYCLLPPKSQLLEYWKSNPSAIPDNVDRRLRKRLMLKKDLRKDDEYNQLARAFKISDVYAPLISSTTSPMTMIQNLNQGEIVYTTTDRVIGARILLYAPRKYYASTLSFTMTKCIIPFGKEVGRAPHSRFNVGTFPSIATPKCFVMSGIDVESIPNEFIKLFYQRVKSVHANILNDISPQILSDMINRKRLRVHTPSDRRAAQLMHLPYHVKRGASHVDVYKVDVVDVLFEVVDVADGLRNVSRKLTMHTVPVCILEMLGIEIADYCVRREDGMFIDWFLLLTMLSDGLTDRRTRCQYLINPSSVPPDVILNISITGFINRHTIDVMPDTYDFVKPIGAVLPKGSFKSTIMRVLDSISILGVQIMPRAHVVDSDEVGEQMEPTFEHAVMEIYKGIAGVDSLDDLIKWVLNSDLIPHDDRLGHLFQAFLPLAKDLLAPMARKFYDNSMSEGRLLTFAHADSELLNANYFGHLLRLKIPYITEVNLMIRKNREGGELFQLVLSHLYKMYATSAQPKWFGSLLRLLICPWLHMEKLIGEADPASTSAEIGWYIPREQLMQDGWCGCEDGFIPYVRIRAPKLVIEELAEKNWGQYHAQVIITDQLVVGEPRRVSAKAVIKGNHLPVKLVSRFACFTLTTKYEMRLSCGHSTGRGAAYNARLAFRSDLA

SEQ ID NO: 18 - protein, 708 aa, Mammalian Orthoreovirus
MGNASSIVQTINVTGDGNVFKPSAETSSTAVPSLSLSPGMLNPGGVPWIAIGDETSVTSPGALRRMTSRDIPETAIINTDNSSGAVPSESALVPYNDEPLVVVTEHAIANFTKAEMALEFNREFLDKLRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDSLIRRYPREAAVALAKRNGGIQWMDVSEGTVMNEAVNAVAASALAPSASSPPLEEKSKLTEQAMDLVTAAEPEIIASLVPVPAPVFAIPPKPADYNVRTLKIDEATWLRMIPKTMTTPFQIQVTDNTSTRWHMNLRGGTRVVNLDQIAPMRFVLDLGGKSYKETSWNPNGKRVGFIVFQSKIPFELWTAASQIGQATVVNYVQLYAEDSSFTDQSIIATTSLAYNYEPEQLNKTDPEMNYYLLATFIDSAEINPSNMTQPDVWDALLTMSPLSAGEVTVKGAVVSEVTPADLVGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQSWTQGFLDKVSTHFPAPRSDCPQSGDSDGGSARRLKRDSYAGVVKRGYTR

SEQ ID NO: 19 - protein, 721 aa, Mammalian Orthoreovirus
MASFKGFSANTVPVSKAKRDISALAATPGFHSQSFTPSVDMSQSREFLTKAIEQGSMSIPYQHVNVPKVDRKVVSLVVRPFSSGAFSISGVISPAHAYLLDCLPQLEQAMAFVASPESFQASDVAKRFAIKPGMSLQDAITAFINFVSAMLKMTVTRQNFDVIVAEIERLASTSVSARTEEAKVADEELMLFGLDHRGPQQLDISDAKGIMKAVDIQTTHDVHLAPGVGNIDPEIYNEGRFMFMQHKPLAADQSYFTLETADYFKIYPTYDEHDGRMADQKQSGLILCTKDEVLAEQTIFKLDTPDDKTVHLLDRDDDHVVARFTKVFIEDVAPGHHTAQRSGQRSVLDDLYANTQVISITSAALKWVVKHGVSDGIVNRKNVKVCVGFDPLYTLSTHNGISLCALLMDEKLSVLNSACRMTLRSLMKTRRDADAHRAFQRVLSQGYTSLMCYYHPSRKLAYGEVLFPERSNDVVDGIKLQLDASRHCHECPVLQQKVVELEKQIVMQKSIQSDTAPIALQPLLSQLRELSSEVTRLQMELSRAQSLNAQLEADVKSAQSCSLDMYLRYHTCINGHAKEDELLDAVRVAPDVRRQIMDRRSEVRKGWCERISKEASAECQTVIDELTLMNGKQAQEIIELRDSAENYEKQIAELVSTITQNQITYQQELQALVAKNVELDTLNQRQARSLRITPSLLSVTPTDSVDGAADLIDFSVPTDEL

SEQ ID NO: 20 - protein, 471 aa, Mammalian Orthoreovirus
MDASLISEIRRIVLQLTQSNNGSINKELKEIKEQVEINATDIRNTNLKLDGIRGQVTSISDSIAAIERRLGEVDGRFVVIANQITQLSNSVGQNTQDISALGDRLNVVEPRIDDLDTFASNLVGRTSTLEADVRGIRTELSALTTRVTTEVARLDALISAGQSSLTSLSSRLDAVEAAMITTAGRGLRKDGNTLNIIVGDGMWFDNSNQLQLDLSNQAKGVGFEGGGVVAKIDTNYFSYNSNGEITLVGQINGLPARVSTLESLKIDTVLPPLTVSEADGARNLGLGYETDDFTVTNSMLSLRSRLTLPTYRYPLELDASINRVQIAERFGMRVGSWTGQLQYQHPQLNWQANVTINLMKVDDWLVLSFTQMTTSGIMADGKFTINFSAGLSSGWQTGETQPSSITDPLSTTFAAVQYLKANGTRVDAFRIMGVSEWVDGELEIKNYGGTYTGHTQVYWAPWTIMYPCNVR

SEQ ID NO: 21 - protein, 418 aa, Mammalian Orthoreovirus
MARAAFLFKTVGFGGLQNVPINDELSSHLLRAGNSPWQLTQFLDWISLGRGLATSALVPTAGSRYYQMSCLLSGTLQIPFRPNHRWGDARFLRLVWSAPTLDGLVVAPPQVLAQPALQAQADRVYDCDDYPFLARDPRFKHRVYQQLSAVTLLNLTGFGPISYVRVDEAMWSGDVNQLLMNYFGHTFAEIAYTLCQASANRPWEHDGTYARMTQIILSLFWLSYVGVIHQQNTYRTFYFQCNRRGDAAEVWILSCSLNHSAQIRPGNRSLFVMPTSPDWNMDVNLILSSTLTGCLCSSSQLPLIDNNSVPAVSRNIHGWTGRAGNQLHGFQVRRMVTEFCDRLRRDGVMTQAQQNQVEALADQTQQFKRDQLEAWAREDDQYNQAHPNSPMFRTKPFTNAQWGRGNTGATSAAIAALI

SEQ ID NO: 22 - protein, 366 aa, Mammalian Orthoreovirus
MASSLRAAISKIKRDDVGQQVCPNYVMLRSSVTTKVVRNVVEYQIRTGGFFSCLAMLRPLQYAKRERLLGQRNLERISTRDILQTRDLHSLCMPTPDAPMSSHQAVTMRELICSYFKVDHTDGLKYIPMDEKYSPSSLARLFTMGMAGLHITTEPSYKRVPIMHLAADLDCMTLALPYMITLDGDTVVPVAPTLSAEQLLDDGLKGMACMDISYGCEVDANNRSAGDQSMDSSRCINELYCEETAEAICVLKTCLVLNCMQFKLEMDDLAHNAAELDKIQMMIPFSERVFRMASAFATIDVQCFRFCVMMKDKNLKIDMRETMRLWTRSALDESVVTSSLSISLDRGRWVAADTNDARLLVFPIRV

SEQ ID NO: 23 - protein, 365 aa, Mammalian Orthoreovirus
MEVCLPNGHQIVDWINNAFEGRVSIYSAQQGWDKTISAQPDMMVCGSAVVCMHCLGVVGSLQRKLKHLPHHKCNQQLREQDYVDLQFADRVTAHWKRGMLSFVSQMHAVMNDVTPEELERVRTDGGILAELDWLKVESGSMFRSIHSSWTDPLQVVEDLDTQLDRYWTALNLMIDSSDLVPNFMMRDPSHAFNGVKLEGEARQTQFPRTFDSGSNLKWGVMVYDYSELERDSQKGRSYRKEIVTPAKDFGHFGLSHYSRATTPILGKMPAVFSGMLTGNCKMYPFIKGTAKLKTVKKLVDAVNHTWGFEKIRYALGPGGMTGWYNRTMQQAPIVLTPAALTMFPDMTRFGDLQYPITIGDPAVLG

SEQ ID NO: 24 - protein, 122 aa, Mammalian Orthoreovirus
MGRSTRNSKKSKNKSRSTLLISGIPILSSMGSEDKLLASVIASQPLNVDWVKWMVDLWLSRIRLRNYLTQLARTLKTYPPWVTDSMLLNQELMTWIHSHLISLDEHPLWRQMLEESVLNCPH

SEQ ID NO: 25 - protein, 681 aa, Mammalian Orthoreovirus
MSQSREFLTKAIEQGSMSIPYQHVNVPKVDRKVVSLVVRPFSSGAFSISGVISPAHAYLLDCLPQLEQAMAFVASPESFQASDVAKRFAIKPGMSLQDAITAFINFVSAMLKMTVTRQNFDVIVAEIERLASTSVSARTEEAKVADEELMLFGLDHRGPQQLDISDAKGIMKAVDIQTTHDVHLAPGVGNIDPEIYNEGRFMFMQHKPLAADQSYFTLETADYFKIYPTYDEHDGRMADQKQSGLILCTKDEVLAEQTIFKLDTPDDKTVHLLDRDDDHVVARFTKVFIEDVAPGHHTAQRSGQRSVLDDLYANTQVISITSAALKWVVKHGVSDGIVNRKNVKVCVGFDPLYTLSTHNGISLCALLMDEKLSVLNSACRMTLRSLMKTRRDADAHRAFQRVLSQGYTSLMCYYHPSRKLAYGEVLFPERSNDVVDGIKLQLDASRHCHECPVLQQKVVELEKQIVMQKSIQSDTAPIALQPLLSQLRELSSEVTRLQMELSRAQSLNAQLEADVKSAQSCSLDMYLRYHTCINGHAKEDELLDAVRVAPDVRRQIMDRRSEVRKGWCERISKEASAECQTVIDELTLMNGKQAQEIIELRDSAENYEKQIAELVSTITQNQITYQQELQALVAKNVELDTLNQRQARSLRITPSLLSVTPTDSVDGAADLIDFSVPTDEL